(19) United States
(12) Patent Application Publication
(10) Pub. No.: US 2026/0364528 A1
(43) Pub. Date: Nov. 19, 2026

(54) ORTHO-HALOGEN-SUBSTITUTED 9,9-BIS(4-AMINOPHENYL)FLUORENE BASED POLYIMIDE MEMBRANES FOR GAS SEPARATION APPLICATIONS

(71) Applicant: Saudi Arabian Oil Company, Dhahran (SA)

(72) Inventors: Ali Hayek, Dhahran (SA); Abdulkarim Alsamah, Dhahran (SA); Rashed H. Alhajry, Dammam (SA)

(21) Appl. No.: 19/195,903

(22) Filed: May 1, 2025

(51) Int. Cl. B01D 53/22; B01D 67/00; B01D 71/64; B01D 71/80; C07C 7/144; C08G 73/10

(52) U.S. Cl. CPC B01D 53/228; B01D 67/0013; B01D 71/64; B01D 71/80; C07C 7/144; C08G 73/1067; B01D 2256/245; B01D 2257/102; B01D 2257/108; B01D 2257/11; B01D 2257/304; B01D 2257/504

(57) ABSTRACT

The present disclosure provides a method for separating methane (CH₄) from a CH₄-containing gas mixture using a membrane. The present disclosure also provides the membrane comprising a polyimide polymer comprising a monomer having a structure of Formula (I), and methods of making the membrane.

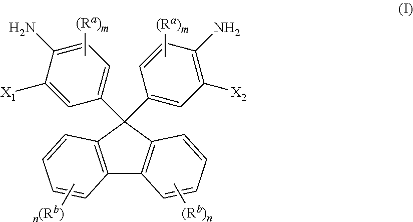

### TECHNICAL FIELD

The present disclosure relates to methods for methane (CH₄) separation using polymer membranes. More specifically, the polymer membranes include polyimide polymers based on an ortho-halogen-substituted 9,9-bis(4-aminophenyl)fluorene (CARDO). The present disclosure also relates to polyimide polymer membranes for sweet and/or sour natural gas purification applications.

### BACKGROUND

Natural gas is a clean, reliable, and affordable energy source that can help reduce greenhouse gas emissions and meet growing energy demands. However, many natural gas reserves contain high levels of acid gases such as hydrogen sulfide (H₂S) and carbon dioxide (CO₂), i.e., sour gas, which can corrode pipelines and pose significant safety risks. To make natural gas suitable for transport and sale, it must undergo a sweetening process. This makes the purification of sour natural gas with high concentrations of H₂S and CO₂ a costly challenge.

Polyimide membranes functionalized with groups such as alkyl, amine, or acyl have been developed; however, these membranes often face a trade-off between permeability and selectivity and do not exhibit both favorable diffusivity and solubility, thus limiting their applications in natural gas separation and purification.

Accordingly, there is a need for polyimide-based membranes that can be used for both sweet and sour natural gas purification applications.

### SUMMARY

Provided in the present disclosure are methods for separating methane CH₄ from a CH₄-containing gas mixture. The method includes providing a membrane comprising a polyimide polymer comprising a monomer having a structure of Formula (I):

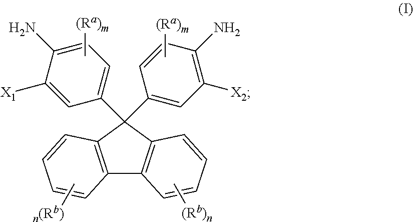

and contacting a first side surface of the membrane with the CH₄-containing gas mixture, thereby allowing the CH₄ to pass through the membrane via a second side surface opposite the first side surface to form a CH₄ gas stream.

In some embodiments, X₁ and X₂ are each independently selected F, Cl, Br, and I. In some embodiments, R^{a} and R^{b} are each independently selected from H, D, CN, halo, C₁₋₆ alkyl, C₂₋₆ alkenyl, C₂₋₆ alkynyl, C₁₋₆ haloalkyl, C₁₋₆ alkoxy, C₁₋₆ haloalkoxy, C₆₋₁₀ aryl, C₃₋₁₄ cycloalkyl, 5-14 membered heteroaryl, 4-14 membered heterocycloalkyl, C₆₋₁₀ aryl-C₁₋₄ alkyl-, C₃₋₁₄ cycloalkyl-C₁₋₄ alkyl-, (5-14 membered heteroaryl)-C₁₋₄ alkyl-, (4-14 membered heterocycloalkyl)-C₁₋₄ alkyl-, NR^{c}R^{c}, NR^{c}C(O)R^{c}, and NR^{c}C(O)OR^{c}, wherein the C₁₋₆ alkyl, C₂₋₆ alkenyl, C₂₋₆ alkynyl, C₁₋₆ haloalkyl, C₁₋₆ alkoxy, C₁₋₆ haloalkoxy, C₆₋₁₀ aryl, C₃₋₁₄ cycloalkyl, 5-14 membered heteroaryl, 4-14 membered heterocycloalkyl, C₆₋₁₀ aryl-C₁₋₄ alkyl-, C₃₋₁₄ cycloalkyl-C₁₋₄ alkyl-, (5-14 membered heteroaryl)-C₁₋₄ alkyl-, and (4-14 membered heterocycloalkyl)-C₁₋₄ alkyl- of R^{a} and R^{b} are each optionally substituted with 1, 2, 3, 4 or 5 independently selected R^{c} substituents. In some embodiments, R^{a} and R^{b} taken together with the C atom to which they are attached form a 6-14 membered aromatic group, or 6-14 membered heteroaromatic group, wherein the 6-14 membered aromatic group or the 6-14 membered heteroaromatic group are optionally substituted with 1, 2, 3, or 4 independently selected R^{c} substituents. In some embodiments, each R^{c} is independently selected from H, D, halo, C₁₋₆ alkyl, C₁₋₆ alkoxy, CN, OH, oxo, NH₂, NHC₁₋₆ alkyl, and N(C₁₋₆ alkyl)₂. In some embodiments, m is an integer selected from 0, 1, 2, and 3; n is an integer selected from 0, 1, 2, 3, and 4.

The method further comprises preparing the membrane comprising the polyimide polymer by obtaining an ortho-halogen-substituted 9,9-bis(4-aminophenyl)fluorene monomer of Formula (I); reacting the ortho-halogen-substituted 9,9-bis(4-aminophenyl)fluorene monomer with a dianhydride monomer to form the polyimide polymer; dissolving the polyimide polymer in a solvent to form a polymer solution; forming a film from the polymer solution; and drying the film to form the membrane.

Also provided in the present disclosure are membranes comprising a polyimide polymer comprising a monomer having a structure of Formula (I).

### BRIEF DESCRIPTION OF THE DRAWINGS

FIG. 1 is a schematic representation of the solution-diffusion process across a polymeric membrane, according to certain embodiments of the present disclosure.

FIG. 2 is a general synthetic scheme for forming an ortho-halogen-substituted 9,9-bis(4-aminophenyl)fluorene (CARDO) moiety, according to certain embodiments of the present disclosure.

FIG. 3 illustrates drawings of chemical structures for symmetric ortho-halogen-substituted CARDO diamine monomers, according to certain embodiments of the present disclosure.

FIG. 4 illustrates drawings of chemical structures for asymmetric ortho-halogen-substituted CARDO diamine monomers, according to certain embodiments of the present disclosure.

FIG. 5 is a general synthetic scheme for forming ortho-halogen-substituted, CARDO-based homopolyimides, according to certain embodiments of the present disclosure.

FIG. 6 is a synthetic scheme for forming ortho-halogen-substituted, CARDO-based co-polyimides, according to certain embodiments of the present disclosure.

FIG. 7 illustrates drawings of chemical structures for dianhydride monomers, according to certain embodiments of the present disclosure.

FIG. 8 illustrates drawings of chemical structures for aromatic diamine comonomers, according to certain embodiments of the present disclosure.

FIG. 9 is a synthetic scheme for forming 4,4′-(9H-fluorene-9,9-diyl)bis(2-bromoaniline) (BCARDO) as described in Example 1, according to certain embodiments of the present disclosure.

FIG. 10 is a synthetic scheme for forming 4,4′-(2,7-di-tert-butyl-9H-fluorene-9,9-diyl)bis(2-bromoaniline) [BCARDO(t-Bu)] as described in Example 2, according to certain embodiments of the present disclosure.

FIG. 11 is a synthetic scheme for forming the homopolymer 6FDA-BCARDO as described in Example 3, according to certain embodiments of the present disclosure.

FIG. 12 is a synthetic scheme for forming the (1:1) block copolyimide 6FDA-mPDA/BCARDO as described in Example 4, according to certain embodiments of the present disclosure.

FIG. 13 is a synthetic scheme for forming the (1:1) block copolyimide 6FDA-DAM/BCARDO as described in Example 5, according to certain embodiments of the present disclosure.

FIG. 14 is a synthetic scheme for forming the (1:1) block copolyimide 6FDA-Durene/BCARDO as described in Example 7, according to certain embodiments of the present disclosure.

FIG. 15 is a ¹H-NMR spectrum of 4,4′-(9H-fluorene-9,9-diyl)bis(2-bromoaniline) (BCARDO) in DMSO-d₆, according to certain embodiments of the present disclosure.

FIG. 16 is a ¹H-NMR spectrum of 6FDA-DAM/BCARDO (1:1) copolyimide in chloroform-d, according to certain embodiments of the present disclosure.

FIG. 17 is a ¹H-NMR spectrum of 4,4′-(2,7-di-tert-butyl-9H-fluorene-9,9-diyl)bis(2-bromoaniline) [BCARDO(t-Bu)] in chloroform-d, according to certain embodiments of the present disclosure.

FIG. 18 is an FTIR spectrum of BCARDO monomer, according to certain embodiments of the present disclosure.

FIG. 19 is an FTIR spectrum of BCARDO(t-Bu) monomer, according to certain embodiments of the present disclosure.

FIG. 20 is a plot of normalized FTIR spectra of 6FDA-BCARDO homopolyimide and a series of BCARDO-based copolyimides at different composition ratios, according to certain embodiments of the present disclosure.

FIG. 21 is a plot of the thermogravimetric analysis (TGA) curves and the first derivative curves (DTG) of the 6FDA-BCARDO homopolyimide and a series of BCARDO-based copolyimides at different composition ratios, according to certain embodiments of the present disclosure.

FIG. 22 is a plot of the differential scanning calorimetric (DSC) curves for the 6FDA-BCARDO homopolyimide and a series of BCARDO-based copolyimides at different composition ratios, according to certain embodiments of the present disclosure.

FIG. 23 is a plot comparing the CO₂/CH₄ permeability-selectivity “trade-off” curve of Robeson to a series of 6FDA-CARDO homopolyimides and a series of BCARDO-based copolyimides at different composition ratios, according to certain embodiments of the present disclosure.

FIG. 24 is a plot showing a comparison between sweet mixed-gas CO₂ permeability (columns) and CO₂/CH₄ selectivity (curves) coefficients of 6FDA-mPDA/BCARDO (1:1) at different feed pressures and 55 °C, according to certain embodiments of the present disclosure.

FIG. 25 is a plot showing a comparison between sweet mixed-gas CO₂ permeability (columns) and CO₂/CH₄ selectivity (curves) coefficients of 6FDA-DAM/BCARDO (1:1) at different feed pressures and 22 °C, according to certain embodiments of the present disclosure.

FIG. 26 is a plot showing a comparison between sweet mixed-gas CO₂ permeability (columns) and CO₂/CH₄ selectivity (curves) coefficients of 6FDA-DAM/BCARDO (1:2) at different feed pressures and 55 °C, according to certain embodiments of the present disclosure.

FIG. 27 is a plot showing a comparison between sweet mixed-gas CO₂ permeability (columns) and CO₂/CH₄ selectivity (curves) coefficients of 6FDA-DAM/BCARDO (1:1) at different temperatures and a feed pressure of 500 psi, according to certain embodiments of the present disclosure.

FIG. 28 is a plot showing a comparison between sweet mixed-gas CO₂ permeability (columns) and CO₂/CH₄ selectivity (curves) coefficients of 6FDA-DAM/BCARDO (1:2) at different temperatures and a feed pressure of 900 psi, according to certain embodiments of the present disclosure.

FIG. 29 is a method for separating methane (CH₄) from a CH₄-containing gas mixture, according to certain embodiments of the present disclosure.

FIG. 30 is a method for preparing a membrane comprising a polyimide polymer, according to certain embodiments of the present disclosure.

### DETAILED DESCRIPTION

Current natural gas purification technology primarily relies on liquid amines to remove acid gases from sub-quality natural gas reserves. However, this approach requires an energy-intensive regeneration step for the liquid amines, increasing operational costs. As a result, researchers worldwide have been exploring alternative technologies to replace amine-based processes. One energy-efficient alternative is the use of gas separation membranes.

To effectively compete with or complement the existing technology, these membranes must meet specific performance criteria in terms of gas permeability (or permeance) for H₂S and CO₂, as well as selectivity for CO₂/CH₄ and H₂S/CH₄. Currently available commercial membranes exhibit CO₂/CH₄ and H₂S/CH₄ selectivity values ranging from 15 to 25, and permeance values of up to 80 GPU for CO₂ and H₂S. Therefore, membrane materials that demonstrate high permeability for CO₂ and H₂S hold significant potential to reduce both capital expenditures (CAPEX) and operating expenditures (OPEX) in membrane-based bulk acid gas removal processes.

As separation technologies continue to evolve, gas separation using polymeric membranes has garnered increasing attention due to advantages such as enhanced energy efficiency, reduced equipment footprint, and lower capital costs compared to conventional separation methods. Although existing membrane technologies may not yet surpass the performance of traditional absorption systems, hybrid systems that integrate both absorption and membrane-based separation have emerged as promising alternatives. Accordingly, there remains a need for polymeric membranes that exhibit improved separation performance to enhance the efficiency and applicability of membrane-based gas separation technologies.

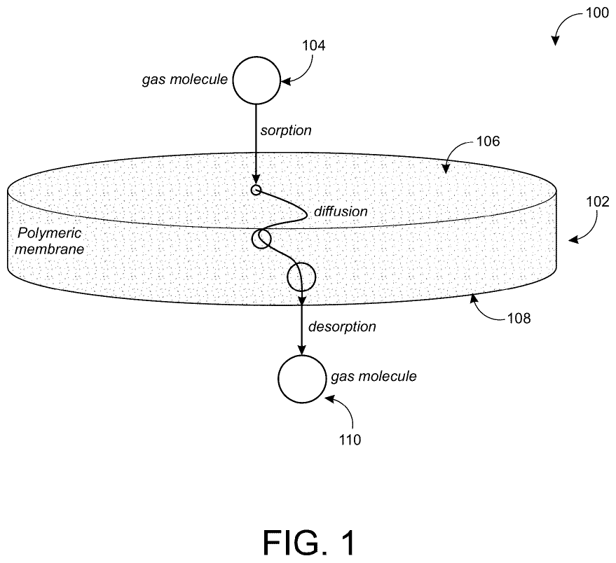

Polymeric membranes are thin, semipermeable barriers that enable the selective separation of certain gas molecules from others. Unlike porous filtration membranes that separate compounds based on molecular size, dense polymeric membranes operate primarily according to the “solution-diffusion” model, as depicted in FIG. 1. In this mechanism, gas molecules first sorb into the surface of the polymeric membrane material, then diffuse through the dense membrane matrix, and ultimately desorb on the downstream side. The efficiency of separation is governed by the differential solubility and diffusivity of individual gas species within the polymer, enabling selective permeation based on molecular interactions rather than size exclusion.

FIG. 1 is a schematic drawing 100 of the solution-diffusion process across a polymeric membrane 102. In this process, a gas molecule 104 sorbs into the membrane polymeric 102 via a first side surface 106, then diffuses through the thin membrane matrix, to end up desorbing 110 from a second side surface 108 opposite the first side surface 106 of the polymeric membrane 102.

Membrane polymers, methods for synthesizing the membrane polymers and forming membranes from the polymers, and methods for using the membranes for CH₄ separation are provided in examples herein. The polymers are based on ortho-halogen-substituted 9,9-bis(4-aminophenyl)fluorene (CARDO) moieties. The membranes formed from these polymers are intended to enhance the performance of polymeric membranes in natural gas separation applications. These materials aim to improve membrane productivity, separation efficiency, and resistance to plasticization under elevated pressures and temperatures encountered during sweet and/or sour gas mixed-gas separation. As a result, the use of such membranes may lead to reduced capital and operational expenditures in membrane-based natural gas purification processes.

Numerous polymeric membranes for gas separation have been developed in the decades, however, only a limited number have been commercialized for sour gas separation applications. Examples of polymeric materials used to form gas separation membranes include, but are not limited to, cellulose acetate (CA), polyimides (PI), and perfluoropolymers, such as polytetrafluoroethylene (PTFE), perfluorocycloalkene (PFCA), and the like. These polymeric materials are generally semi-crystalline and possess a glass transition temperature (T_{g}) of greater than about 100 °C.

The chemical structure of the polymeric material is a key factor in the selection and design of membranes for gas separation applications. Various classes of polymers have been investigated for use in membranes intended for sour mixed-gas separation, including polyimides and polyoxadiazoles. An effective polymeric material should form membranes that exhibit high permeability coefficients for CO₂ and H₂S, along with high to moderate selectivity coefficients for CO₂/CH₄ and H₂S/CH₄ gas pairs. Additionally, the material must be capable of withstanding the harsh chemical, physical, and thermal environments typically encountered during natural gas purification.

One way of improving the membrane performance is by tailoring the properties of the polymeric material forming the membrane by (1) increasing the dynamic free volume of the membrane by adding bulky groups or a heteroatom (i.e., halogen) with larger size than carbon atom, and/or (2) improving the gas-polymer affinity by increasing the polarity within the polymer backbone. Tailoring may also be used to improve the gas-polymer affinity by increasing the polarity within the polymer backbone.

This methodology can be used to allow the polymeric material to deal with each type of gas molecule in a distinct manner. For example, the dynamic free volume within the membrane matrix can separate the gas molecules based on the difference of their sizes (i.e., kinetic diameters, Dₖ) according to a kinetic phenomenon (i.e., rate of diffusion), since methane (Dₖ=3.80 Å, the main component of natural gas) is larger in size than the undesired existent impurities in natural gas (i.e., Dₖ=3.30 Å for CO₂ and Dₖ=3.60 Å for H₂S). Further, improving the gas-polymer affinity increases the solubility of polar gas molecules, such as H₂S, or molecules containing polar bonds, such as CO₂, to improve the permeation through the membrane matrix, while not influencing the transport of methane.

Halogen atoms, such as fluorine (F, atomic radii=64 pm; electronegativity=3.98), chlorine (Cl, atomic radii=99 pm; electronegativity=3.16), bromine (Br, atomic radii=114 pm; electronegativity=2.96) and iodine (I, atomic radii=133 pm; electronegativity=2.66) are known for their increased polarity and most are larger than carbon atoms (C, atomic radii=77 pm; electronegativity=2.55), with the exception of fluorine, which is smaller in size than carbon, but with much higher electronegativity. The polar nature of halogen atoms increases the affinity of acid gases to polymeric chains (H₂S is a polar molecule, and CO₂ is a non-polar molecule with polar bonds). Thus, adding halogen atoms to the polymer backbone leads to fine-tune the diffusivity and solubility of gas molecules by increasing the dynamic free volume within the membrane matrix and the gas-polymer affinity, respectively. The techniques described herein improve the sweet and sour mixed-gas separation properties of polymeric membranes and their resistance to plasticization at elevated feed pressures, by developing ortho-halogen-substituted CARDO-based polyimides.

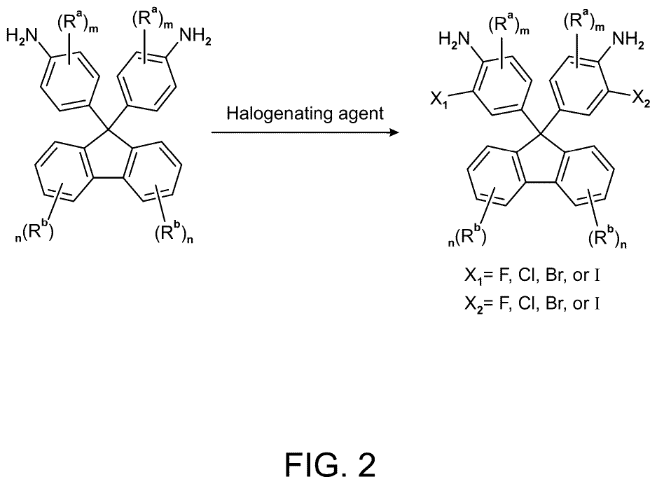

FIG. 2 is a general synthetic scheme for forming an ortho-halogen-substituted 9,9-bis(4-aminophenyl)fluorene (CARDO) moiety. The diamine ortho-halogenated CARDO monomer is of an aromatic type, which means the primary amine groups are located on aromatic rings connected directly or through covalent bonds to the fluorene central moiety. The halogenation reaction occurs selectively at the ortho position of the primary amine groups as illustrated in FIG. 2.

This disclosure describes the preparation of polyimides structures containing CARDO-based diamine monomers containing at least one halogen atom at the ortho positions to their primary amine groups for applications in purification of sour natural gas streams. The halogen atoms are introduced at least one C₂ position of the phenyl ring of the 9,9-bis(4-aminophenyl)fluorene (CARDO) moiety to serve as polar bulky groups, which can simultaneously improve the solubility and diffusivity of gas molecules through the polymeric matrix of their membranes. Additionally, their bulkiness may disrupt interchain interactions, thereby improving gas permeation without significantly compromising the membrane's selectivity.

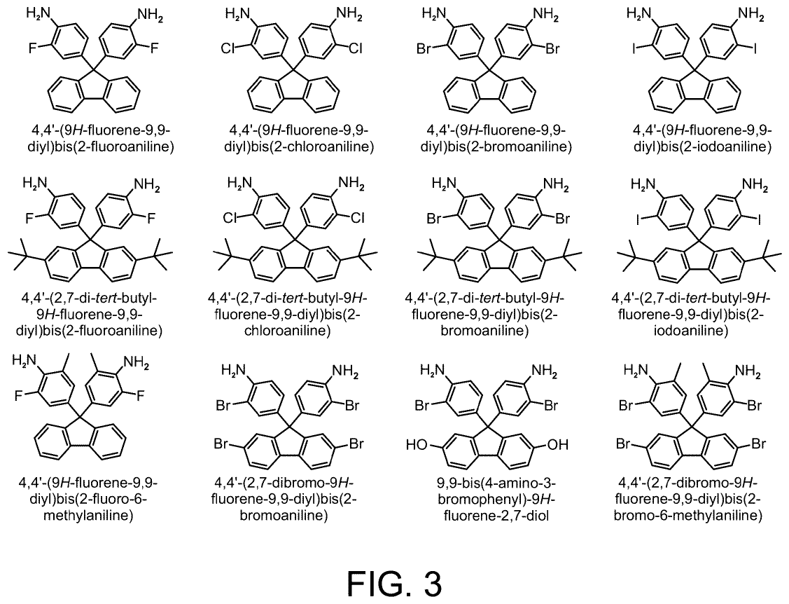

The halogen substitution at the C₂ position of the CARDO moiety may be either symmetrical or asymmetrical. FIG. 3 depicts chemical structures of symmetric ortho-halogen-substituted CARDO diamine monomers. In some embodiments, the ortho-halogen-substitution of the CARDO moiety is symmetric, e.g., X₁=X₂. In these structures, both X₁ and X₂ are halogen atoms.

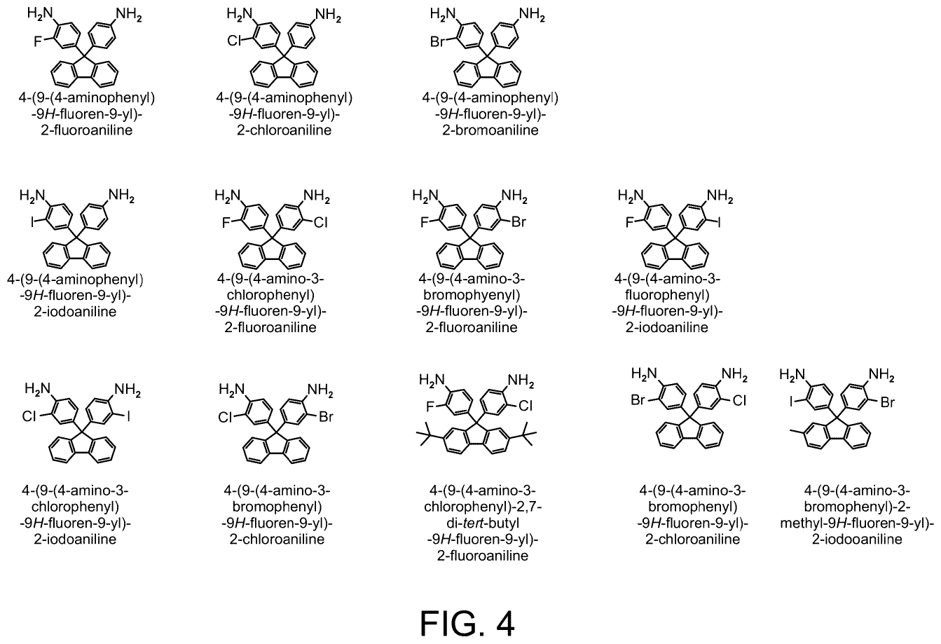

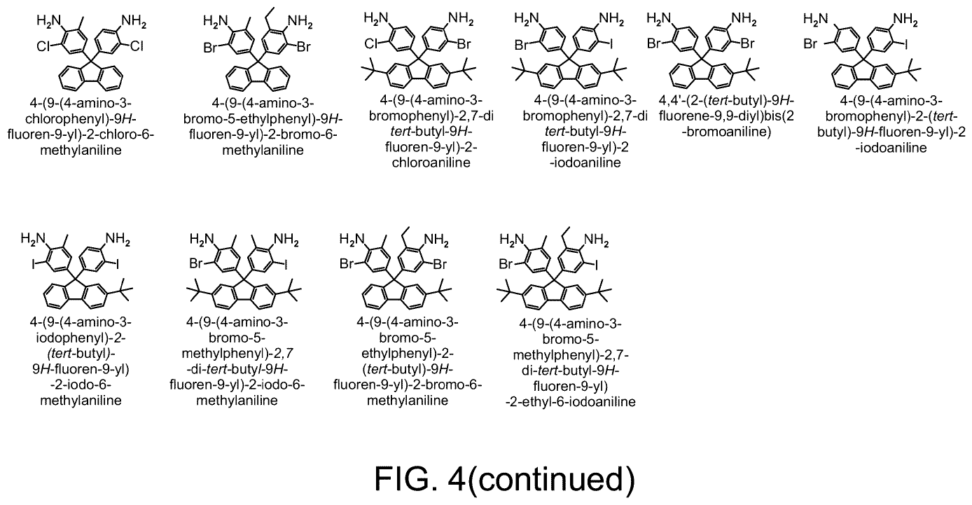

FIG. 4 depicts chemical structures of asymmetric halogen-substituted CARDO diamine monomers. In some embodiments, the halogen-substitution of the CARDO moiety is asymmetric, e.g., X₁≠X₂, examples of which are shown in FIG. 4. For these structures, one of X₁ or X₂ can be a hydrogen atom, with the other being a halogen atom, or both of them are different halogen atoms. The fluorenyl ring of the CARDO diamine monomer may be either unsubstituted or substituted. Substitutions can occur at one or multiple available positions on the fluorenyl ring and may include various atoms (e.g., hydrogen, halogen) or functional groups (e.g., alkyl, aryl, amine derivatives).

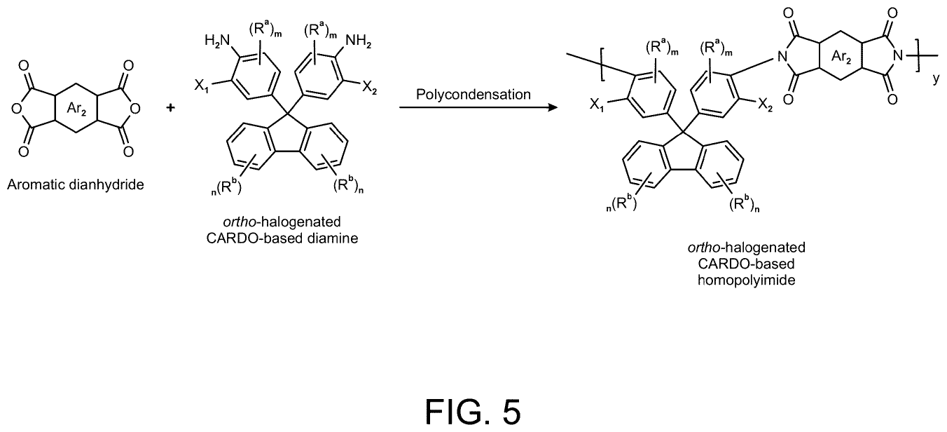

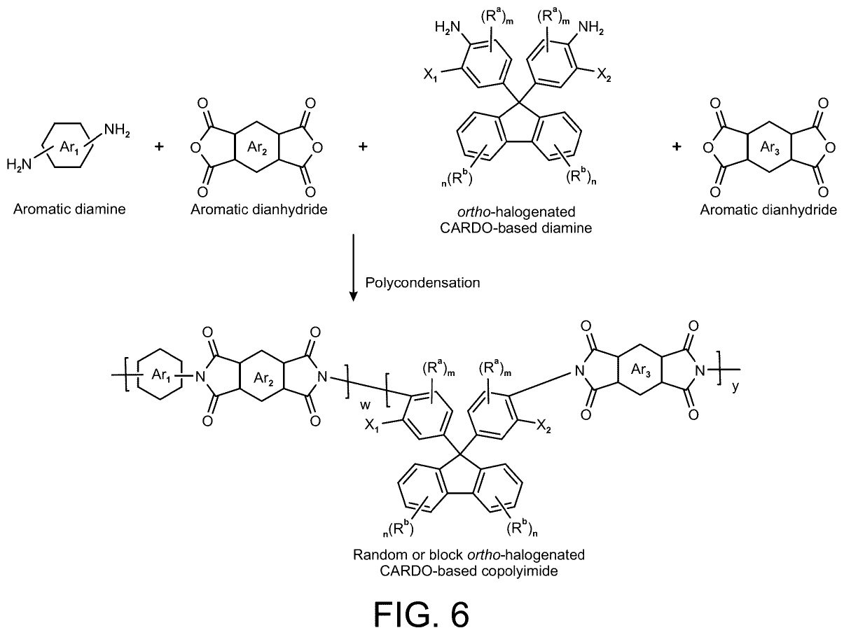

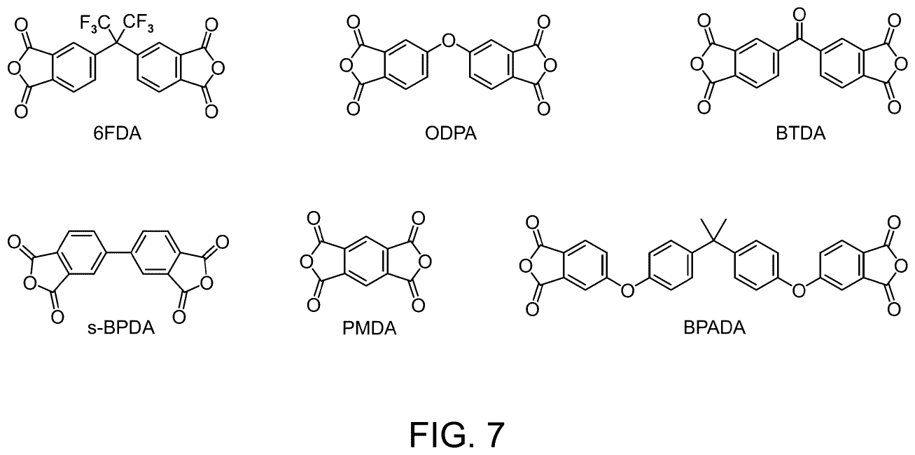

The prepared ortho-halogen-substituted CARDO moieties are reacted with a variety of dianhydride moieties to form polymers, such as homopolyimides, as shown in FIG. 5, or copolyimides, as shown in FIG. 6. In some embodiments, both symmetric and asymmetric ortho-halogen-substituted CARDO moieties are used to form the polymers, allowing the separation properties of the membrane to be tailored. In some embodiments, the dianhydride includes, but is not limited to those as depicted in FIG. 7.

FIG. 5 is a general synthetic scheme for forming ortho-halogen-substituted, CARDO-based homopolyimides. The reaction may be carried out in m-cresol at an elevated temperature (180 °C-200 °C). Other solvents may be used, such as N-methyl pyrrolidone (NMP) and dimethylacetamide (DMAc).

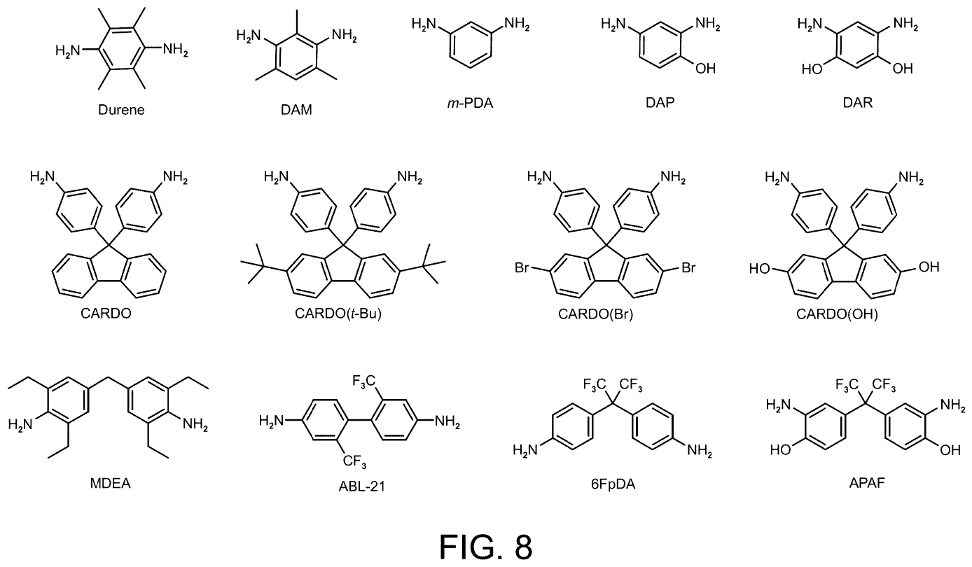

FIG. 6 is a synthetic scheme for forming ortho-halogen-substituted, CARDO-based co-polyimides. The copolyimides are prepared by using an ortho-halogen-substituted CARDO moiety and a dianhydride monomer as shown in FIG. 7. In some embodiments, the copolyimides are prepared in the presence of one or more additional diamine comonomers, e.g., as shown in FIG. 8.

The copolymerization is a synthetic methodology that aims to combine the gas permeation properties of two separate homopolymers into a single copolymer. The second diamine comonomer is chosen to complement the properties provided by the ortho-halogen-substituted CARDO-based homopolyimide to improve the permeability and/or selectivity of the copolyimide membrane.

As described herein, for natural gas purification, it is useful to improve the permeability of impurities [such as carbon dioxide (CO₂) and hydrogen sulfide (H₂S)] and the membrane selectivities (CO₂/CH₄ and H₂S/CH₄) toward the main hydrocarbon constituting the natural gas-methane (CH₄). In another aspect, it is desired to increase the plasticization resistance of polymeric membranes during high-pressure mixed-gas separation. The halogen polar groups are intended to form a dipole-dipole type interaction between the polymeric chains, which limits their mobility under harsh separation conditions of temperature and pressure. This strategy enhances the permeation properties and plasticization resistance of the polymeric membranes during mixed-gas separation under high feed pressure conditions.

FIG. 7 depicts drawings of chemical structures for dianhydride monomers. Various dianhydride monomers can be used to form the polymers and copolymers described herein, including, for example, but not limited to those shown in FIG. 7.

FIG. 8 depicts drawings of chemical structures for aromatic diamine comonomers. For the preparation of the copolyimides described in this disclosure, various diamine comonomers are combined with the dianhydride and ortho-halogen-substituted CARDO monomers can be used to provide polymers segments to tailor the properties of the targeted polymeric materials. The aromatic diamine monomers may include, but not limited to, any of the structures listed in FIG. 8.

In some embodiments, ortho-hydroxyl-containing co-monomers are combined with the dianhydride and ortho-halogen-substituted CARDO monomers in the preparation of the copolyimides described in this disclosure. In some embodiments, the ortho-hydroxyl-containing co-monomers include, but are not limited to, diaminophenol (DAP), diaminoresorcinol (DAR), 2,2-Bis [4-(4-aminophenoxy)phenyl] hexafluoropropane (APAF), or any combinations thereof. In some embodiments, the ortho-hydroxyl-containing co-monomers can enhance the thermal rearrangement of ortho-halogenated CARDO-based polymers. This is not limited to the examples listed above but may extend to any chemical structure that facilitates such thermal rearrangement.

This disclosure also encompasses other thermal or chemical crosslinking strategies that may be employed to tailor the gas separation properties of ortho-halogenated CARDO-based polymers.

As described herein, the synthetic methodology allows the preparation of a large variety of new polymers, including but not limited to, homopolymers, random copolymers, block copolymers, terpolymers, and so on. The polymers formed are used to prepare polymeric membranes with improved sweet or sour mixed-gas separations properties for natural gas purification application. The term sour means that the gas stream contains high levels of H₂S, where membranes are prone to plasticization due to the high affinity of H₂S molecules to polymeric materials due to the polar nature of H₂S molecules. The changes can be implemented at every stage of the preparation of the polymer.

### EXAMPLES

### Materials

All materials listed in this work were used as received. Aniline (ACS reagent, ≥99.5%), sodium sulfite anhydrous (purity ≥98.0%), and toluene (ACS reagent, ≥99.5%) were purchased from Sigma-Aldrich, USA. 4,4′-(hexa-fluoroisopropylidene)diphthalic anhydride (6FDA, purity 99.0%), and m-cresol (purity 99.0%) were acquired from Alfa Aesar. 2,3,5,6-tetramethylbenzene-1,4-diamine (Durene; purity 98.0%), and 2,7-dibromo-9H-fluoren-9-one (purity >98.0%) were obtained from TCI America. Methanol (purity 98.0%), ethyl acetate (reagent grade), and N,N-dimethylacetamide (DMAc, reagent grade) were purchased from Fisher Scientific. Sodium hydroxide (purity >98.0%), and 2-propanol (purity >99.9%) were acquired from Fluka.

### Characterization Techniques

### Proton Nuclear Magnetic Resonance Spectroscopy (¹H-NMR)

The ¹H-NMR spectra of the prepared polymers and monomers were recorded using a JEOL 500 MHz NMR spectrometer in deuterated chloroform (CHCl₃-d) or dimethyl sulfoxide (DMSO-d₆) accordingly.

### Fourier Transform Infra-Red Spectroscopy (FTIR)

The FTIR spectra were recorded using a Thermo Scientific Nicolet iS50 spectrometer in transmission mode. Samples could be of the form of powder solid or membrane.

### Thermogravimetric Analysis (TGA) and Glass Transition Temperature (T_{g})

The TGA plots and the differential scanning calorimetry (DSC) traces were performed using a NETZSCH STA 449 F3 Jupiter®. The TGA plots were recorded at a temperature range from 30 °C to 650 °C with a heating rate of 10 °C/min under a nitrogen atmosphere. The T_{g} was determined from the DSC traces over two consecutive cycles. Each cycle consists of heating the sample at a temperature range between 30 °C and 450 °C, using a heating rate of 10 °C/min under a nitrogen flow. The first run is aimed to clear the thermal history of the sample, and the T_{g} values were determined after the second cycle.

### Syntheses of Monomers and Polymers

### Example 1: Preparation of 4,4′-(9H-fluorene-9,9-diyl)bis(2-bromoaniline) (BCARDO)

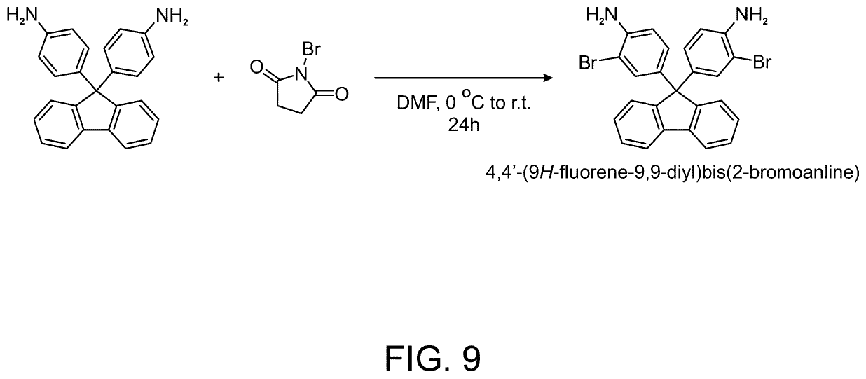

FIG. 9 illustrates a synthetic scheme for preparing 4,4′-(9H-fluorene-9,9-diyl)bis(2-bromoaniline) (BCARDO) of the present disclosure. In a 250 mL three-neck round-bottom flask, equipped with an addition funnel and under a nitrogen atmosphere, 4,4′-(9H-fluorene-9,9-diyl)dianiline (CARDO, 10.0 g, 28.7 mmol) was dissolved in 100 mL of DMF, and the reaction mixture was cooled down to 0 °C in an ice bath. Then, a solution of N-bromosuccinimide (10.22 g, 57.4 mmol) in 30 mL of DMF was added dropwise at 0 °C. The reaction mixture was allowed to warm up to room temperature slowly while keeping the ice bath in place and allowed to stir overnight. The reaction mixture was then poured into a separation funnel containing an aqueous solution of 1 M sodium hydroxide (100 mL) and ethyl acetate (100 mL). The organic layer was isolated and dried over magnesium sulfate. After removal of the solvent, 4,4′-(9H-fluorene-9,9-diyl)bis(2-bromoaniline) (14.53 g, 28.7 mmol, quantitative yield) was obtained as a light yellow powder. For purification, the monomer was recrystallized from toluene at 90 °C, filtered and dried to obtain a white powder. ¹H-NMR (500 MHz, DMSO-d₆) δ 7.94-7.83 (m, 2H), 7.43-7.25 (m, 6H), 6.89 (dq, J=4.1, 2.1 Hz, 4H), 6.70 (d, J=9.0 Hz, 2H), 5.24 (s, 4H).

### Example 2: Preparation of 4,4′-(2,7-di-tert-butyl-9H-fluorene-9,9-diyl)bis(2-bromoaniline) [BCARDO(t-Bu)]

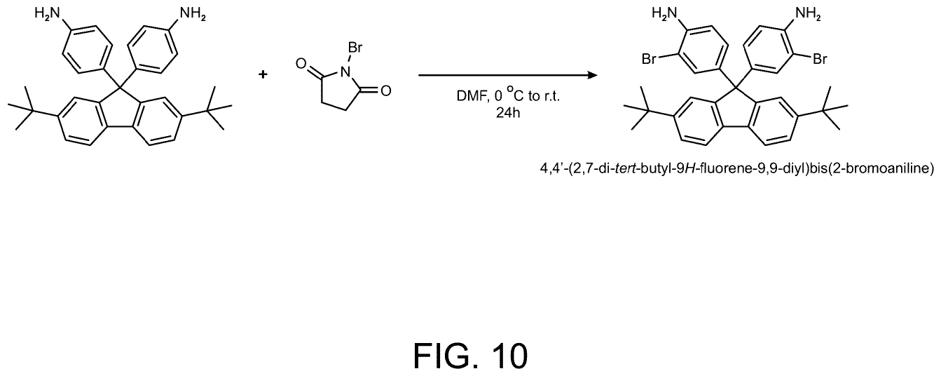

FIG. 10 illustrates a synthetic scheme for preparing 4,4′-(2,7-di-tert-butyl-9H-fluorene-9,9-diyl)bis(2-bromoaniline) [BCARDO(t-Bu)] of the present disclosure. In a 250 mL three-neck round-bottom flask, equipped with an addition funnel and under a nitrogen atmosphere, 4,4′-(2,7-di-tert-butyl-9H-fluorene-9,9-diyl)dianiline (2.5 g, 5.43 mmol) was dissolved in 30 mL of DMF, and the reaction mixture was cooled down to 0 °C in an ice bath. Then, a solution of N-bromosuccinimide (1.932 g, 10.85 mmol) in 10 mL of DMF was added dropwise at 0 °C. The reaction mixture was allowed to warm up to room temperature slowly while keeping the ice bath in place and allowed to stir overnight. The reaction mixture was then poured into a separation funnel containing an aqueous solution of 1 M sodium hydroxide (100 mL) and ethyl acetate (100 mL). The organic layer was isolated and dried over magnesium sulfate. After removal of the solvent, 4,4′-(2,7-di-tert-butyl-9H-fluorene-9,9-diyl)bis(2-bromoaniline) (3.02 g, 4.88 mmol, 90% yield) was obtained as a brown powder. ¹H-NMR (500 MHz, Chloroform-d) δ 7.61 (d, J=8.4 Hz, 2H), 7.36 (d, J=5.9 Hz, 4H), 7.17 (s, 2H), 6.97 (dd, J=8.4, 2.2 Hz, 2H), 6.64 (d, J=8.4 Hz, 2H), 3.98 (s, 4H), 1.30 (s, 18H).

### Example 3: Preparation of Homopolymer 6FDA-BCARDO

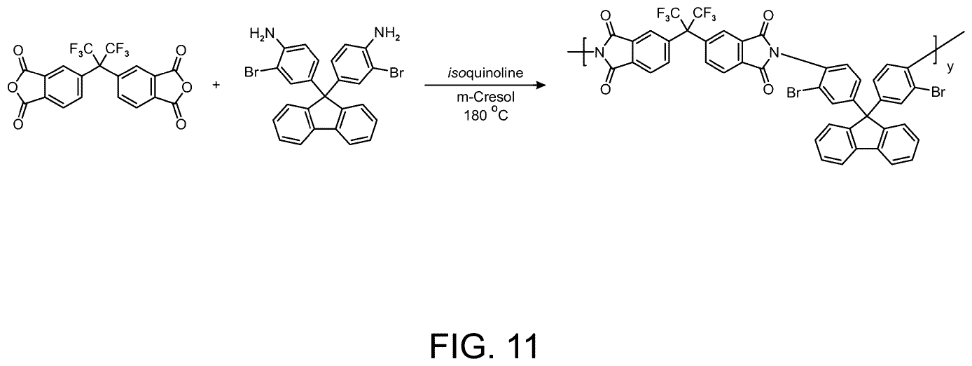

FIG. 11 illustrates a synthetic scheme for preparing the 6FDA-BCARDO of the present disclosure. In a 100-mL three-neck round bottom flask equipped with a nitrogen inlet and a mechanical stirrer, 4,4′-(9H-fluorene-9,9-diyl)bis(2-bromoaniline) (1.000 g, 1.975 mmol) (BCARDO) and 5,5′-(perfluoropropane-2,2-diyl)bis(isobenzofuran-1,3-dione) (0.878 g, 1.975 mmol) (6FDA) were added to the reaction vessel followed by 10.0 mL of m-cresol and 10 drops of isoquinoline. The mixture was then heated at 85 °C for 48 hours. The temperature was later increased to 150 °C for 4 hours, then to 180 °C for another 4 hours. Heat was removed, and the reaction mixture was allowed to cool down below 100 °C, then the resulting highly viscous solution was poured into methanol in thin fibers. The fibrous polymer obtained was ground, rinsed with methanol, filtered and dried under reduced pressure for 24 h at 60 °C to afford 6FDA-BCARDO (1.828 g, 1.936 mmol, 98% yield) as a white off powder. ¹H-NMR (500 MHz, DMSO-d₆) δ 8.18 (t, J=6.8 Hz, 2H), 8.05-7.92 (m, 4H), 7.84 (d, J=8.3 Hz, 2H), 7.70 (d, J=7.5 Hz, 2H), 7.60 (d, J=9.0 Hz, 2H), 7.51-7.35 (m, 8H).

### Example 4: Preparation of Block Copolyimide 6FDA-mPDA/BCARDO (1:1)

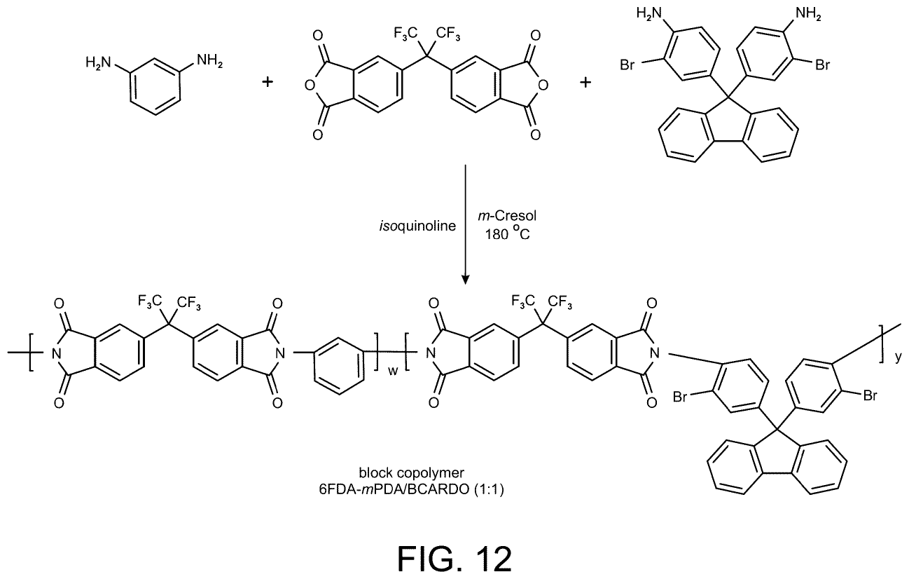

FIG. 12 illustrates a synthetic scheme for preparing the block copolyimide 6FDA-mPDA/BCARDO (1:1) of the present disclosure. In a 100-mL three-neck round bottom flask equipped with a nitrogen inlet and a mechanical stirrer, 5,5′-(perfluoropropane-2,2-diyl)bis(isobenzofuran-1,3-dione) (0.873 g, 1.964 mmol) (6FDA), benzene-1,3-diamine (0.250 g, 2.312 mmol) (mPDA) were introduced and dissolved in 10.0 mL of m-cresol and the reaction mixture was heated to 85 °C, and stirred for 24 hours. Then 5,5′-(perfluoropropane-2,2-diyl)bis(isobenzofuran-1,3-dione) (1.182 g, 2.660 mmol) (6FDA), and 4,4′-(9H-fluorene-9,9-diyl)bis(2-bromoaniline) (1.170 g, 2.312 mmol) (BCARDO) were added to the flask, followed by 5.00 mL of m-cresol and 10 drops of isoquinoline. The stirring continued for an additional 24 hours at 85 °C. Later, the heat was gradually increased to 150 °C and 180 °C for 4 hours each. The heat was removed, and the reaction mixture was allowed to cool down below 100 °C, then the resulting highly viscous solution was poured into methanol in thin fibers. The fibrous polymer obtained was ground, rinsed with methanol four times, filtered, and dried under reduced pressure for 24 h at 60 °C to afford 6FDA-mPDA/BCARDO (1:1) (3.31 g, 2.266 mmol, 98% yield) as a white off powder. ¹H-NMR (500 MHz, chloroform-d) δ 8.04 (t, J=6.7 Hz, 4H), 7.97 (s, 4H), 7.87 (d, J=8.2 Hz, 4H), 7.81 (d, J=7.3 Hz, 2H), 7.65 (d, J=8.0 Hz, 2H), 7.55 (d, J=7.2 Hz, 2H), 7.51 (s, 2H), 7.44 (dd, J=11.3, 7.3 Hz, 4H), 7.38 (m, 4H), 7.24 (t, J=2.9 Hz, 2H).

### Example 5: Preparation of Block Copolyimide 6FDA-DAM/BCARDO (1:1)

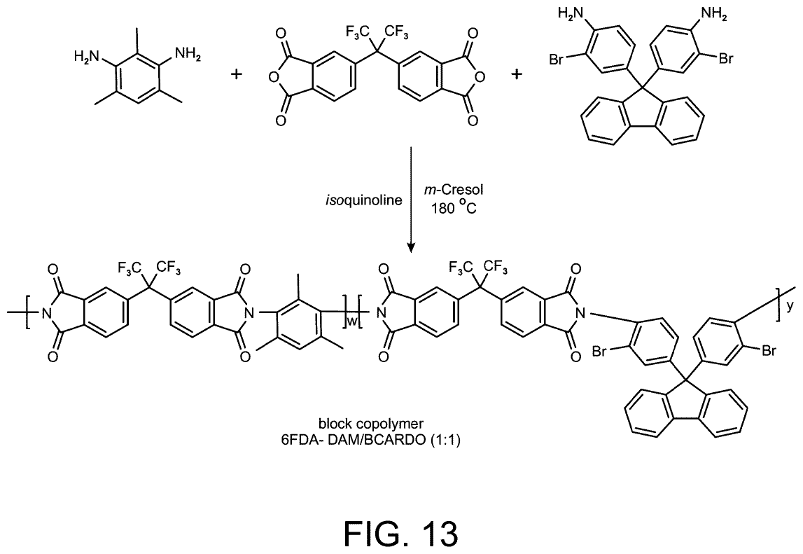

FIG. 13 illustrates a synthetic scheme for preparing the block copolyimide 6FDA-mPDA/BCARDO (1:1) of the present disclosure. In a 100-mL three-neck round bottom flask equipped with a nitrogen inlet and a mechanical stirrer, 5,5′-(perfluoropropane-2,2-diyl)bis(isobenzofuran-1,3-dione) (0.628 g, 1.414 mmol) (6FDA), and 2,4,6-trimethylbenzene-1,3-diamine (0.250 g, 1.664 mmol) (DAM) were introduced and dissolved in 10.0 mL of m-cresol and the reaction mixture was heated to 85 °C, and stirred for 24 hours. Then 5,5′-(perfluoropropane-2,2-diyl)bis(isobenzofuran-1,3-dione) (0.850 g, 1.914 mmol) (6FDA), and 4,4′-(9H-fluorene-9,9-diyl)bis(2-bromoaniline) (0.842 g, 1.664 mmol) (BCARDO) were added to the flask, followed by 5.00 mL of m-cresol and 10 drops of isoquinoline. The stirring continued for an additional 24 hours at 85 °C. Later, the heat was gradually increased to 150 °C and 180 °C for 4 hours each. The heat was removed, and the reaction mixture was allowed to cool down below 100 °C, then the resulting highly viscous solution was poured into methanol in thin fibers. The fibrous polymer obtained was ground, rinsed with methanol four times, filtered, and dried under reduced pressure for 24 h at 60 °C to afford 6FDA-DAM/BCARDO (1:1) (2.451 g, 1.631 mmol, 98% yield) as a white off powder. ¹H-NMR (500 MHz, chloroform-d) δ 8.13-7.73 (m, 14H), 7.52-7.29 (m, 10H), 7.24 (t, J=2.75 Hz, 3H), 2.22 (s, 6H), 1.98 (s, 3H).

### Example 6: Preparation of Block Copolyimide 6FDA-DAM/BCARDO (1:2)

FIG. 13 also illustrates a synthetic scheme for preparing the block copolyimide 6FDA-mPDA/BCARDO (1:2) of the present disclosure. In a 100-mL three-neck round bottom flask equipped with a nitrogen inlet and a mechanical stirrer, 5,5′-(perfluoropropane-2,2-diyl)bis(isobenzofuran-1,3-dione) (0.628 g, 1.414 mmol) (6FDA), 2,4,6-trimethylbenzene-1,3-diamine (0.250 g, 1.664 mmol) (DAM) were introduced and dissolved in 10.0 mL of m-cresol and the reaction mixture was heated to 85 °C, and stirred for 24 hours. Then 5,5′-(perfluoropropane-2,2-diyl)bis(isobenzofuran-1,3-dione) (1.589 g, 3.578 mmol) (6FDA), 4,4′-(9H-fluorene-9,9-diyl)bis(2-bromoaniline) (1.685 g, 3.33 mmol) were added to the flask, followed by 10.00 mL of m-cresol and 10 drops of isoquinoline. The stirring continued for an additional 24 hours at 85 °C. Later, the heat was gradually increased to 150 °C and 180 °C for 4 hours each. The heat was removed, and the reaction mixture was allowed to cool down below 100 °C, then the resulting highly viscous solution was poured into methanol in thin fibers. The fibrous polymer obtained was ground, rinsed with methanol four times, filtered, and dried under reduced pressure for 24 h at 60 °C to afford 6FDA-DAM/BCARDO (1:2) (2.451 g, 1.631 mmol, 98% yield) as a white off powder. ¹H-NMR (500 MHz, Chloroform-d) δ 8.11-7.74 (m, 22H), 7.51 (t, J=2.2 Hz, 4H), 7.47-7.32 (m, 16H), 7.24 (m, 5H), 2.21 (s, 6H), 1.98 (s, 3H).

### Example 7: Preparation of Block Copolyimide 6FDA-Durene/BCARDO (1:1)

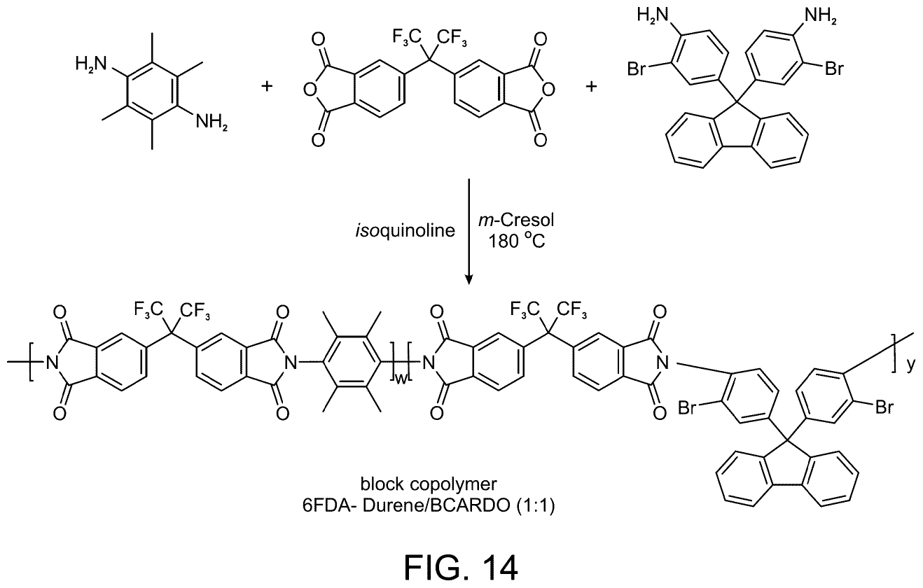

FIG. 14 illustrates a synthetic scheme for preparing the block copolyimide 6FDA-Durene/BCARDO (1:1) of the present disclosure. In a 100-mL three-neck round bottom flask equipped with a nitrogen inlet and a mechanical stirrer, 5,5′-(perfluoropropane-2,2-diyl)bis(isobenzofuran-1,3-dione) (0.574 g, 1.293 mmol) (6FDA), 2,3,5,6-tetramethylbenzene-1,4-diamine (0.250 g, 1.522 mmol) (Durene) were introduced and dissolved in 10.0 mL of m-cresol and the reaction mixture was heated to 85 °C, and stirred for 24 hours. Then 5,5′-(perfluoropropane-2,2-diyl)bis(isobenzofuran-1,3-dione) (0.778 g, 1.751 mmol) (6FDA), and 4,4′-(9H-fluorene-9,9-diyl)bis(2-bromoaniline) (0.770 g, 1.522 mmol) (BCARDO) were added to the flask, followed by 5.00 mL of m-cresol and 10 drops of isoquinoline. The stirring continued for an additional 24 hours at 85 °C. Later, the heat was gradually increased to 150 °C and 180 °C for 4 hours each. The heat was removed, and the reaction mixture was allowed to cool down below 100 °C, then the resulting highly viscous solution was poured into methanol in thin fibers. The fibrous polymer obtained was ground, rinsed with methanol four times, filtered, and dried under reduced pressure for 24 h at 60 °C to afford 6FDA-Durene/BCARDO (1:1) (2.263 g, 1.492 mmol, 98% yield) as a white off powder. ¹H-NMR (500 MHz, chloroform-d) δ 8.12-7.77 (m, 14H), 7.54-7.32 (m, 10H), 7.27-7.17 (m, 2H), 2.14 (s, 12H).

### Chemical Characterization

The various chemical structures of the compounds prepared in this disclosure were confirmed using ¹H-NMR in deuterated solvents. Examples of the spectra for these compounds are illustrated below. Further confirmation was provided by the results from FTIR analysis.

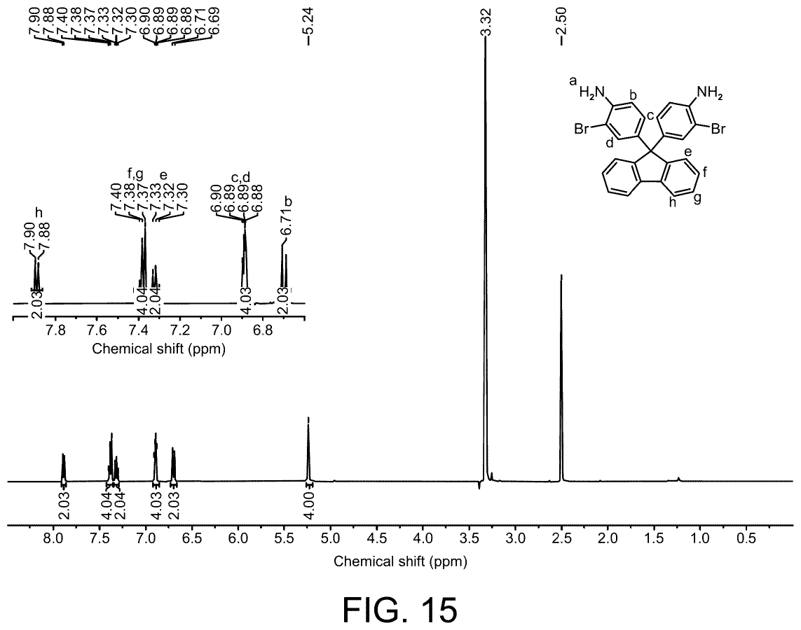

FIG. 15 is a ¹H-NMR spectrum of 4,4′-(9H-fluorene-9,9-diyl)bis(2-bromoaniline) (BCARDO) in DMSO-d₆. The chemical structure of the BCARDO monomer was confirmed by its ¹H-NMR spectrum in deuterated DMSO-d₆ as illustrated in FIG. 15. The spectrum illustrates all representative peaks corresponding to hydrogen atoms a-h of the BCARDO as shown in FIG. 15. The absence of any undesired peak within the spectrum is an indication to the good purity of the prepared polymer.

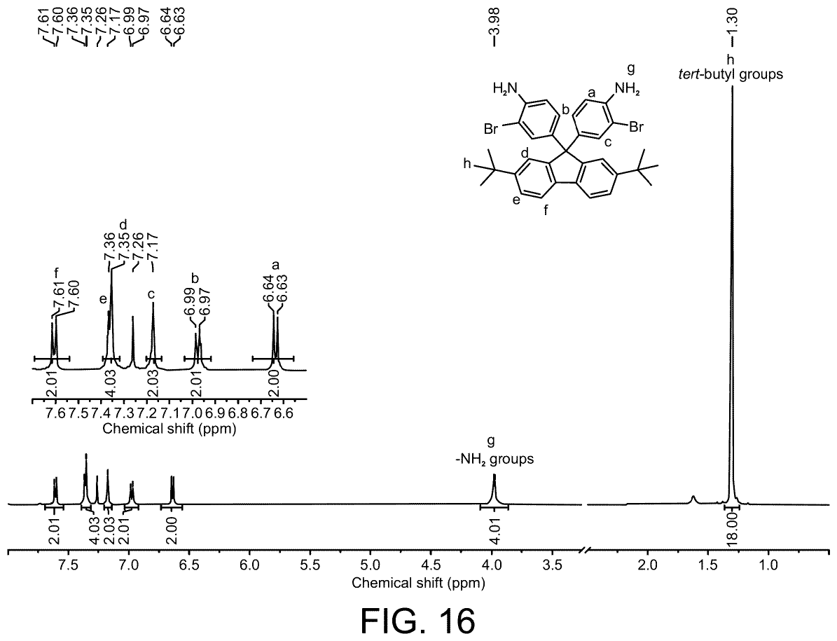

FIG. 16 is a ¹H-NMR spectrum of 4,4′-(2,7-di-tert-butyl-9H-fluorene-9,9-diyl)bis(2-bromoaniline) [BCARDO(t-Bu)] in Chloroform-d. The chemical structure of the BCARDO(t-Bu) monomer was confirmed by its ¹H-NMR spectrum in chloroform-d as illustrated in FIG. 16. The spectrum illustrates all representative peaks corresponding to hydrogen atoms a-h of the BCARDO(t-Bu) as shown in FIG. 16. The absence of any undesired peak within the spectrum is an indication to the good purity of the prepared polymer.

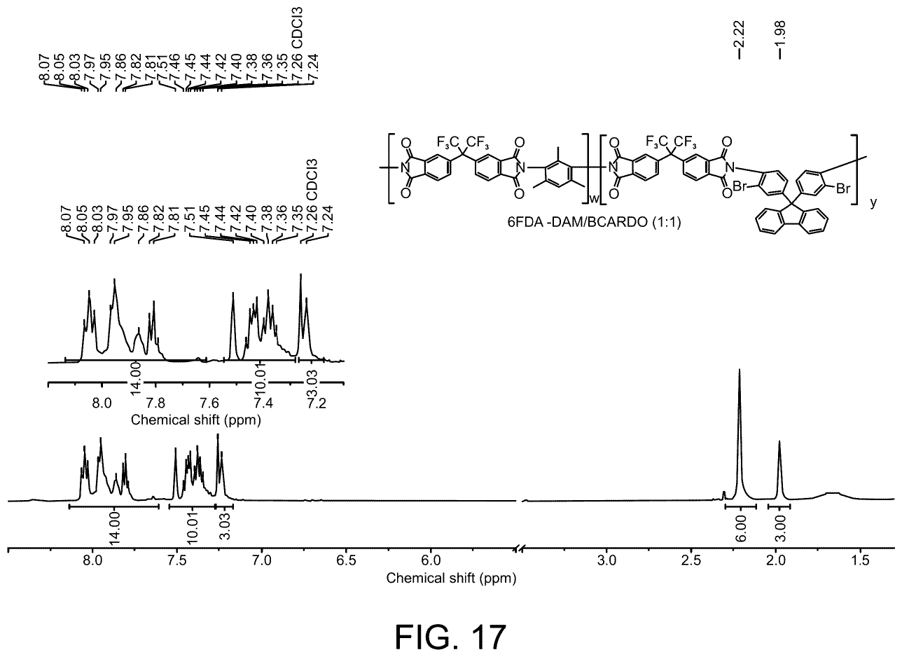

For copolyimides, in addition to determining their chemical structures and purities, the ¹H-NMR spectra are further used to determine the molecular ratio of the co-monomers within the copolymer backbone. For example, the desired molar ratio between the co-monomers DAM and BCARDO in the 6FDA-DAM/BCARDO (1:1) block copolymer described in Example 5, is calculated from the ratio of the integrated peak areas of the aromatic peaks of BCARDO and aliphatic peaks of DAM, from the spectrum illustrated in FIG. 17.

For instance, the DAM monomer possesses one aromatic proton that shows up as a singlet at 7.24 ppm, and 9 aliphatic protons, which appear as two singlets at 1.98 ppm and 2.22 ppm, that correspond to its three methyl groups. As per the spectrum illustrated in FIG. 17, the aromatic peak of DAM overlaps with one of the aromatic peaks of BCARDO at 7.24 ppm. If the total integration is set to 3 protons (1 DAM unit and 1 BCARDO unit), the signals integration of the aliphatic methyl groups of DAM should be equal to 3 protons at 1.98 ppm and 6 protons at 2.22 ppm. The remaining aromatic protons should be equal to 27 protons (1 proton for DAM unit, 12 protons for 6FDA units, and 14 protons for BCARDO unit). The analysis of the ¹H-NMR spectrum of the 6FDA-DAM/BCARDO (1:1) block copolymer indicates a molar ratio of 1:1 between DAM and BCARDO co-monomers. The absence of any undesired peaks within the spectrum is an indication to the good purity of the prepared polymer.

In a similar way, the molar ratios between mPDA, Durene, and DAM and BCARDO comonomers in 6FDA-mPDA/BCARDO (1:1), 6FDA-Durene/BCARDO (1:1) and 6FDA-DAM/BCARDO (1:2) block copolymers (Examples 4, 6, and 7) were determined using their corresponding ¹H-NMR spectra.

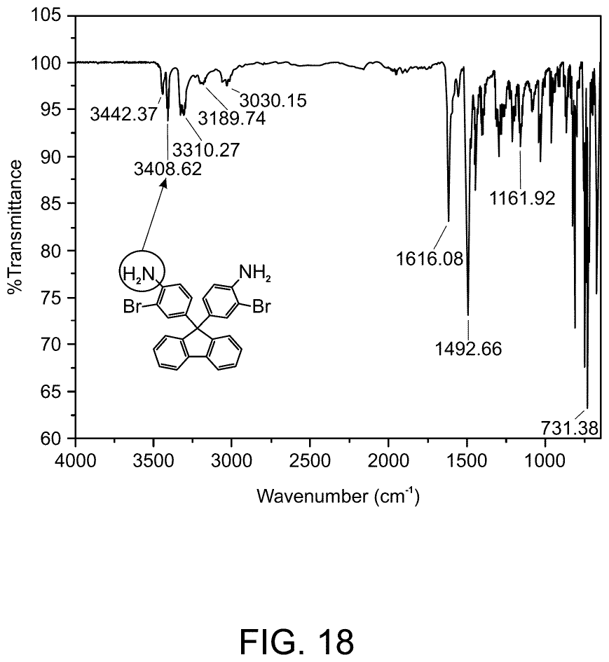

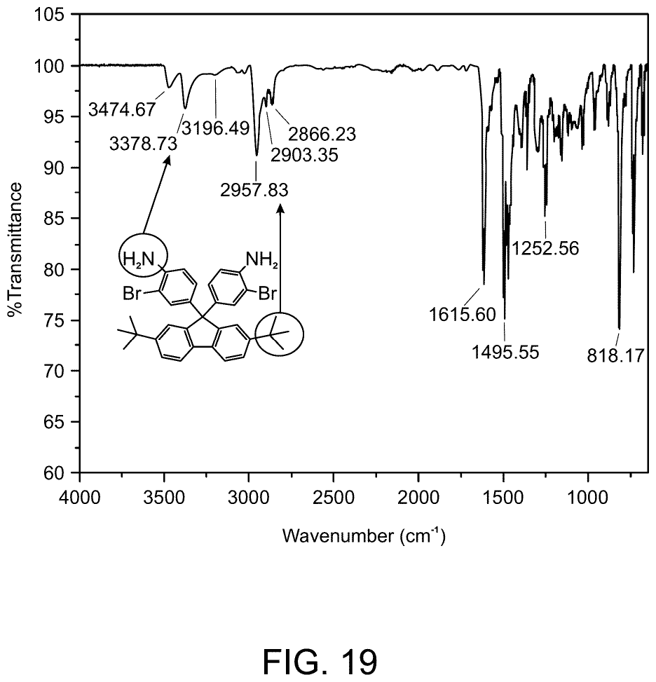

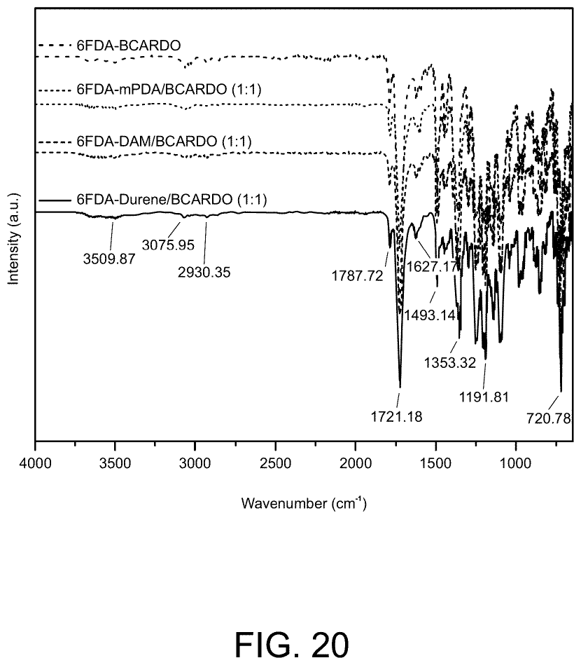

FTIR spectra were recorded for the monomers and polymers disclosed herein, including 6FDA-BCARDO, 6FDA-mPDA/BCARDO, 6FDA-DAM/BCARDO, and 6FDA-Durene/BCARDO, at various composition ratios. The FTIR spectra of the diamine monomers are important to depict the availability of the primary amine groups (—NH₂) as illustrated in FIGS. 18 and 19, and some corresponding functional groups, such as the tert-butyl groups of BCARDO(t-Bu) as illustrated in FIG. 19. The FTIR spectra of copolymers are recoded to ensure that the polycondensation reaction is completed as depicted in FIG. 20. In general, the absence of any peaks that correspond to the intermediate species the polyamic acid (3500-3100 and 1700-1650 cm⁻¹) demonstrates that the reaction is completed. Further, the symmetric and asymmetric carbonyl groups of the imide ring depicted at 1787 and 1721 cm⁻¹, respectively, confirm the formation of the imide ring (final product) within the polymer backbone. In addition, the imide ring deformation band appears at 720 cm⁻¹. Finally, the appearance of the peaks at around 2930 cm⁻¹ in the FTIR spectra of the copolyimides in FIG. 20 are attributed to the aliphatic C—H bonds of the methyl groups in Durene and DAM.

### Thermal and Physical Properties

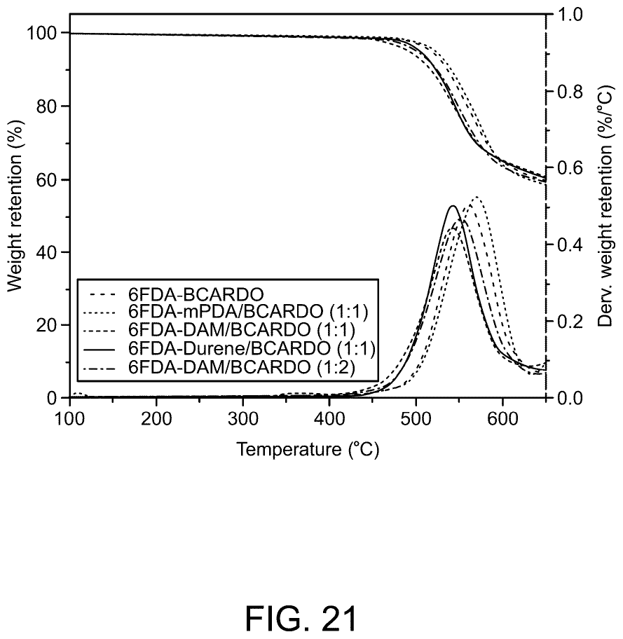

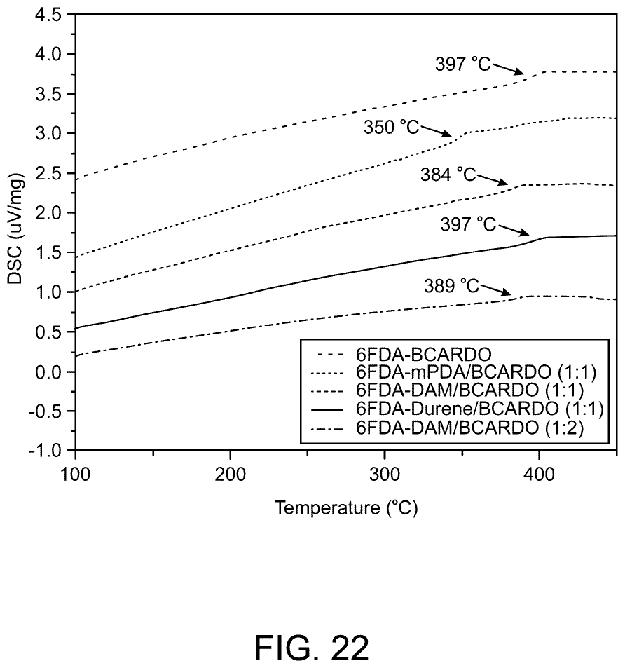

The thermal properties of the prepared polymers—including 6FDA-BCARDO, 6FDA-mPDA/BCARDO, 6FDA-DAM/BCARDO, and 6FDA-Durene/BCARDO—at various composition ratios were measured using TGA and DSC. The results are presented in FIGS. 21 and 22.

FIG. 21 is a plot of the TGA curves and the first derivative curves (DTG) of the prepared polymers. The decomposition temperatures at 5% and 10% were determined (Table 1) to evaluate the thermal stability of the prepared polymers during the harsh industrial conditions of gas separation application. All T_{d5%} of the prepared polymers were found to be higher than 490 °C, which is similar to other highly stable membranes used in gas separation technology. The first derivatives of the TGA curves (FIG. 21) were calculated and the values are listed in Table 1. These values (>540 °C) indicate the highest temperature at which the polymer degrades the fastest, are additional indication to the high thermal stability of the prepared polymers—including 6FDA-BCARDO, 6FDA-mPDA/BCARDO, 6FDA-DAM/BCARDO, and 6FDA-Durene/BCARDO—at various composition ratios.

TABLE 1
Thermal properties of the prepared polymers in this disclosure.

| | DSC | TGA | | |
|---|---|---|---|---|
| | T_{g} | T_{d5%} | T_{d10%} | DTG |
| | (°C) | (°C) | (°C) | (°C) |
| 6FDA-mPDA | 301 | 522 | 541 | 593 |
| 6FDA-DAM | 395 | 516 | 530 | 545 |
| 6FDA-Durene | 426 | 510 | 527 | 542 |
| 6FDA-BCARDO | 397 | 520 | 537 | 562 |
| 6FDA-mPDA/BCARDO (1:1) | 350 | 522 | 542 | 570 |
| 6FDA-DAM/BCARDO (1:1) | 384 | 492 | 517 | 543 |
| 6FDA-Durene/BCARDO (1:1) | 397 | 506 | 524 | 544 |
| 6FDA-DAM/BCARDO (1:2) | 389 | 504 | 524 | 551 |

FIG. 22 is a plot of the differential scanning calorimetric (DSC) curves for the prepared polymers—including 6FDA-BCARDO, 6FDA-mPDA/BCARDO, 6FDA-DAM/BCARDO, and 6FDA-Durene/BCARDO—at various composition ratios. The T_{g} of the prepared polymers were calculated from their corresponding DSC traces and the values are listed in Table 1. To obtain the T_{g} values, polymer samples were heated for two cycles. The first heating cycle was used to clear the thermal history of the polymer, where the T_{g} was recorded after the second heating cycle. All the recorded T_{g} values were greater than 300 °C. These high temperatures indicate the rigidity of the polymeric chains, which is correlated to their performance during gas separation testing. The values obtained are similar to other glassy polymers used in gas separation technology.

### Membrane Preparation

The polymeric membranes were prepared using a solution casting method. Solutions with concentration of 3 wt. % polymer in N,N-dimethylformamide (DMF) were prepared. Then, 11 mL of the solution were filtered through a 0.45 μm PTFE filter to remove any possible solid impurities and poured into a leveled 5.5 cm diameter flat glass Petri dish. The casting dish was placed in a preheated oven at 90 °C, under a gentle flow of nitrogen to allow a slow evaporation of the solvent. After 24 hours, the obtained membrane was further dried at 200 °C under vacuum for another 24 hours. The membrane was peeled off from the Petri dish by soaking in deionized water for a few minutes before peeling from the Petri dish. The membranes were then dried at 60 °C under vacuum for 6 hours. The thickness of the prepared membranes was determined to be in the range of 80-100 μm. For an individual membrane, the standard deviation of thickness uniformity was less than 3%. The formed membrane was cut using a 4 cm diameter cutter, to fit into the membrane cell of the gas permeation testing system.

### Permeation Measurements

A continuous-flow gas permeation device was used for measuring single gas and mixed gas permeation properties. A test gas cylinder provides a single gas or gas mixture for testing. A pressure transducer measures the gas pressure from the test gas cylinder. An actuated valve allows the test gas to flow through or pressurize the interior of a sample membrane that is surrounded by a chamber. A second actuated valve allows retentate samples to be taken from the inside of the sample membrane as opposed to permeate samples from the chamber. A pressure transducer measures the pressure of the permeate in the chamber.

A mass flow meter measures the amount of permeate exiting the chamber. A sample collector can be used to collect samples of permeate or retentate for analysis. A pressure transducer is used to measure the pressure of gas in the sample collector. A carrier gas cylinder provides a carrier gas, such as helium, for a gas chromatograph. The flow rate of the carrier gas is set by a needle valve. After collection of a gas sample in the sample collector, other valves are closed, and an actuated valve is opened to sweep the sample to the gas chromatograph. A vacuum pump is used to pull a vacuum on the system before and between test runs.

### Pure-Gas Permeation Measurement

The pure-gas permeation properties of the prepared polymeric membranes were measured using an in-house built constant volume/variable pressure permeation system. The membrane was placed in the permeation cell and subjected to a selected gas feed (i.e., He, N₂, CH₄ and CO₂) for a specific time, to reach a permeation steady-state at a constant feed pressure of 100 psi and a temperature of 22 °C. A separation membrane can be evaluated by its two intrinsic key characteristics: permeability (P) and selectivity (a). The permeability defines the productivity of the membrane, while the selectivity represents its efficiency. The permeability coefficients (P) were calculated from the slope

$${( \frac{{dp}_{p}}{dt} )}_{ss}$$

of the steady state (ss) of the permeate pressure (pₚ) versus time curve using the following equation:

$$P = 1 {0}^{10} \frac{{V}_{d} l}{{p}_{f} A R T} [ {( \frac{{dp}_{p}}{dt} )}_{ss} - {( \frac{{dp}_{p}}{dt} )}_{l e a k} ] \qquad ( 1 )$$

where, V_{d} is the permeate tube volume (cm³), l is the membrane thickness (cm), p_{f} is the gas feed pressure (cmHg), A is the membrane effective surface area (cm²), R is the universal gas constant (R=0.278 cm³·cmHg/cm³(STP)·K), T is the operational temperature (K),

$${( \frac{{dp}_{p}}{dt} )}_{ss}$$

is the steady-state pressure variation in the permeate tube (cmHg), and

$${( \frac{{dp}_{p}}{dt} )}_{l e a k}$$

is the leak rate of the system, which is usually very small and may be neglected. The permeability coefficient is expressed in Barrer, where 1 Barrer=10⁻¹⁰ cm³(STP)·cm/(cm²·s·cmHg).

The ideal selectivity coefficient for two selected gases A and B is calculated from the ratio of their corresponding permeabilities (P_{A} and P_{B}) using the following expression:

$${α}_{A / B} = \frac{{P}_{A}}{{P}_{B}} \qquad ( 2 )$$

The permeability coefficient is governed by two main stages of the gas permeation process: sorption and diffusion. The sorption of a gas penetrant into the membrane, which is a thermodynamic process, depends mainly on the gas properties (condensability and gas-polymer affinity), however, the diffusion, which is a kinetic process, depends on the gas particle size (kinetic diameter). The smaller the kinetic diameter, the higher the diffusion rate. Therefore, the permeability can be defined using the following expression:

P=D×S (3)

where D is the diffusivity coefficient (cm²/s), and S is the solubility coefficient (cm³(STP)·cm/(cm²·s·cmHg)). Using this equation, the ideal selectivity expression could be modified using the solubility and diffusivity coefficients by:

$${α}_{A / B} = \frac{{P}_{A}}{{P}_{B}} = ( \frac{{D}_{A}}{{D}_{B}} ) \times ( \frac{{S}_{A}}{{S}_{B}} ) \qquad ( 4 )$$

The diffusivity coefficients D (cm²/s) of the gas penetrants were calculated by the time-lag method using the following expression:

$$D = \frac{{l}^{2}}{6 θ} \qquad ( 5 )$$

where θ (s) is the time-lag determined from the pure-gas permeability measurement, and l (cm) in the membrane thickness. The solubility coefficient S (cm³(STP)·cm/(cm²·s·cmHg)) can be then calculated from the permeability and diffusivity coefficients using the following equation:

$$S = \frac{P}{D} \qquad ( 6 )$$

### Results of Permeation Tests

The pure-gas permeation properties of membranes prepared from the studied polymers were determined using the permeation system in a constant-volume mode. For tests performed in the present disclosure, four different pure gases were used: He, N₂, CH₄, and CO₂. The polymeric membranes permeability and selectivity coefficients were calculated from the steady state of the pressure versus time curve, using a constant feed pressure of 100 psi and an operating temperature of 22 °C. The obtained results are listed in Table 2. The permeability coefficients are listed in Barrer, where 1 Barrer=10⁻¹⁰ cm³(STP)·cm/(cm²·s·cmHg).

TABLE 2
Pure gas permeability and selectivity coefficients for the polymeric membranes
measured at 100 psi feed pressure and at 22 °C.

| | Permeability | | | | | | |
|---|---|---|---|---|---|---|---|
| | coefficients (Barrer) | | | | Selectivity coefficients | | |
| Polyimide | He | N₂ | CH₄ | CO₂ | He/CH₄ | N₂/CH₄ | CO₂/CH₄ |
| 6FDA-mPDA | 16.8 | 0.150 | 0.0769 | 3.50 | 218 | 1.94 | 45.5 |
| 6FDA-DAM | 332 | 35.0 | 24.3 | 541 | 13.7 | 1.44 | 22.3 |
| 6FDA-Durene | 451 | 55.9 | 46.1 | 740 | 9.77 | 1.21 | 16.0 |
| 6FDA-BCARDO | 148 | 3.53 | 2.02 | 83.7 | 73.2 | 1.74 | 41.4 |
| 6FDA-mPDA/BCARDO (1:1) | 32.3 | 1.70 | 0.835 | 38.5 | 38.7 | 2.03 | 46.2 |
| 6FDA-DAM/BCARDO (1:1) | 165 | 7.07 | 4.31 | 136 | 38.2 | 1.64 | 31.5 |
| 6FDA-Durene/BCARDO (1:1) | 225 | 12.2 | 8.38 | 205 | 26.9 | 1.47 | 24.4 |
| 6FDA-DAM/BCARDO (1:2) | 176 | 6.61 | 4.05 | 133 | 43.4 | 1.63 | 32.8 |

The pure-gas permeation properties of the 6FDA-BCARDO homopolyimide indicate its potential use as a selectivity-enhancing segment in copolyimides, with an ideal CO₂/CH₄ selectivity of 41.4. However, its moderate CO₂ permeability (83.7 Barrer) may limit its standalone application in industrial settings. To address this, 6FDA-BCARDO can be copolymerized with a permeability-enhancing segment such as 6FDA-Durene or 6FDA-DAM. Alternatively, for applications requiring high selectivity at elevated operating temperatures (e.g., 45 °C or 55 °C), copolymerization with another selectivity-enhancing co-monomer, such as mPDA, may be employed to meet performance goals.

Copolymerization is a strategy to enhance the mechanical properties and gas permeation performance of membranes by selecting comonomers with complementary characteristics. Durene (2,3,5,6-tetramethylbenzene-1,4-diamine) and DAM (2,4,6-trimethylbenzene-1,3-diamine) are two such comonomers for producing membranes with favorable mechanical strength and gas transport properties. Accordingly, a series of copolyimides were synthesized with Durene:BCARDO and DAM:BCARDO in molar ratios of 1:1 and 1:2, respectively. Additionally, a high-selectivity copolymer with an mPDA:BCARDO molar ratio of 1:1 was also prepared. The pure-gas permeation properties of the resulting membranes were evaluated, and the results are summarized in Table 2.

As part of the molecular design strategy for new polymeric materials, the Durene moiety was substituted with a DAM moiety to form block copolyimides with DAM:BCARDO molar ratios of 1:1 and 1:2, resulting in 6FDA-DAM/BCARDO (1:1) and 6FDA-DAM/BCARDO (1:2), respectively. The 6FDA-DAM/BCARDO (1:1) copolymer yielded a membrane exhibiting approximately 29% higher CO₂/CH₄ selectivity compared to the corresponding 6FDA-Durene/BCARDO (1:1) membrane, although with an approximately 34% lower CO₂ permeability.

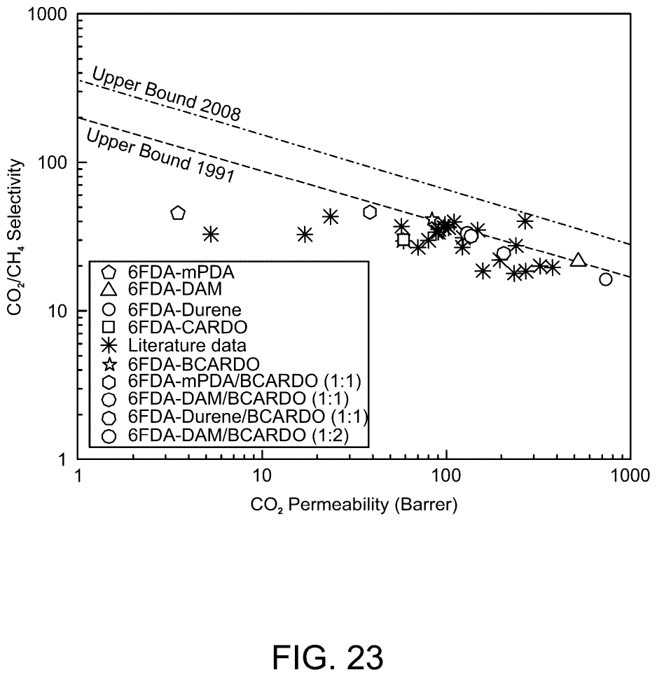

FIG. 23 is a plot comparing the CO₂/CH₄ permeability-selectivity “trade-off” curve of Robeson to the present polymers. In the early 1990s, a research survey determined that polymers with a high selectivity have a low permeability and that the opposite is also true, that materials with a low selectivity have a high permeability. This provides the plot line labeled as “Upper Bound 1991.” The research survey was updated to reflect advancements in membrane technology in an article in 2008, providing the plot line labeled “Upper Bound 2008.” See L. M. Robeson, “The Upper Bound Revisited,” Journal of Membrane Science 320, 390-400 (2008).

In general, membranes fabricated from glassy polymers exhibit a well-known trade-off between permeability and selectivity, as illustrated in FIG. 23. For instance, the CO₂/CH₄ selectivity decreased from 46.2 in 6FDA-mPDA/BCARDO (1:1) to 24.4 in 6FDA-Durene/BCARDO (1:1), while the CO₂ permeability increased from 38.5 Barrer to 205 Barrer, respectively. Notably, the gas permeation properties of the ortho-brominated CARDO-containing polymeric membranes achieved a balance of permeability and selectivity that falls within a commercially favored range.

To better understand the separation process of the prepared membranes, the CO₂ and CH₄ diffusivity coefficients (in cm³(STP)·cm/(cm²·s·cmHg)) were measured using the time-lag method. The obtained results are listed in Table 4.

TABLE 3
CO₂ and CH₄ diffusivity coefficients of
prepared polymers at 100 psi and 22 °C.

| | Diffusivity | | Diffusivity |
|---|---|---|---|
| | (cm²/s) × 10⁻⁸ | | Selectivity |
| Polymer | CH₄ | CO₂ | CO₂/CH₄ |
| 6FDA-BCARDO | 0.428 | 2.62 | 6.12 |
| 6FDA-mPDA/BCARDO (1:1) | 0.398 | 2.98 | 7.49 |
| 6FDA-DAM/BCARDO (1:1) | 0.866 | 8.26 | 9.54 |
| 6FDA-Durene/BCARDO (1:1) | 1.42 | 11.0 | 7.75 |
| 6FDA-DAM/BCARDO (1:2) | 1.06 | 7.98 | 7.53 |

The diffusivity coefficients for both CO₂ and CH₄ in the DAM:BCARDO copolyimide series were observed to decrease as the DAM molar ratio in the copolymer backbone was reduced.

Since the permeability coefficient (P) is calculated from the product of diffusivity (D) and solubility(S) coefficients, the solubility coefficients of the prepared polymers were calculated using equation (9). The obtained solubility coefficients [in cm³(STP)·cm/(cm²·s·cmHg)] are listed in Table 4.

TABLE 4
CO₂ and CH₄ solubility coefficients of
prepared polymers at 100 psi and 22 °C.

| | Solubility (cm³(STP)/ | | Solubility |
|---|---|---|---|
| | cm³cmHg) × 10⁻² | | Selectivity |
| Polymer | CH₄ | CO₂ | CO₂/CH₄ |
| 6FDA-BCARDO | 4.72 | 31.9 | 6.76 |
| 6FDA-mPDA/BCARDO (1:1) | 2.10 | 12.9 | 6.14 |
| 6FDA-DAM/BCARDO (1:1) | 4.97 | 16.5 | 3.32 |
| 6FDA-Durene/BCARDO (1:1) | 5.90 | 18.6 | 3.15 |
| 6FDA-DAM/BCARDO (1:2) | 3.83 | 16.7 | 4.36 |

The CO₂/CH₄ diffusivity and solubility selectivity coefficients were calculated and the results are listed in Table 3 and Table 4, respectively. The CO₂/CH₄ diffusivity selectivity coefficients of the prepared polyimides were higher than the CO₂/CH₄ solubility selectivity coefficients, indicating that the separation through these polymeric membranes is diffusivity driven.

### Mixed-Gas Permeation Measurement

Since natural gas is a mixture of different gases, it is desirable to measure the mixed-gas separation performance of the polymeric membranes. The permeation of gases through glassy polyimide membranes is affected by the presence of other gases in the flow. It is known that a small partial pressure of a condensable species such as CO₂ in the feed gas can significantly reduce the permeability of a gas relative to its permeability individually. Therefore, the competition for Langmuir sorption sites for non-plasticized polymers will lead to a decrease of all penetrants permeabilities. Moreover, the selectivity coefficients may also decrease because of bulk flow and the change on the dynamic free volume (plasticization). Therefore, the permeability of a gas A in the mixed gas permeation process is given by:

$${P}_{A} = {P}_{total} \frac{{y}_{A} ( {p}_{p} - {p}_{f} )}{{x}_{A} {p}_{p} - {y}_{A} {p}_{f}} \qquad ( 7 )$$

Where Pₜₒₜₐₗ is the permeability of total gas particles permeated through the membrane, x_{A} and y_{A} are the mole fractions of gas A in the feed and the permeate sides, respectively, and are determined experimentally using a gas chromatography analyzer connected to the system. The terms pₚ and p_{f} are the partial pressures of gas A in the feed and the permeate sides, respectively. Pₜₒₜₐₗ can be determined using the following expression:

$${P}_{total} = \frac{Jl}{Δ p} \qquad ( 8 )$$

where, J is the penetrant flux (cm³(STP)·cm/(cm²·s·cmHg)), l is the membrane thickness (cm) and Δp is the difference between the partial pressures of gas A at the feed and the permeate sides (cmHg).

The selectivity coefficient (α*_{A/B}) which is the ability of a polymeric membrane to separate a binary feed gas mixture, is defined as follows:

$${α}_{A / B}^{*} = ( \frac{{y}_{A}}{{y}_{B}} ) \times ( \frac{{x}_{B}}{{x}_{A}} ) \qquad ( 9 )$$

where y_{A} and y_{B} are the mole fractions of gases A and B at the permeate side, and x_{A} and x_{B} are the mole fractions of gases A and B at the feed side.

To reflect the real properties of the membrane in the case of a non-ideal gas mixture, the modified expression of the selectivity

$$( {α}_{A / B}^{m , *} )$$

is represented by,

$${α}_{A / B}^{m , *} = \frac{{P}_{A}^{*}}{{P}_{B}^{*}} \qquad ( 10 )$$

where

$${P}_{A}^{*} and {P}_{B}^{*}$$

are the mixed gas permeability coefficients of components A and B determined by the fugacity driving force definition.

### Gas Feed Pressure Effect

Given that natural gas consists of a mixture of various gases, it is important to evaluate the mixed-gas separation performance of the polymeric membranes. Accordingly, the membranes described in this disclosure were tested using a sweet gas mixture comprising 29.9 mol % CO₂, 60 mol % CH₄, and 10.1 mol % N₂. Permeation measurements were conducted at feed pressures ranging from 300 to 900 psi with an increment of 200 psi at a temperature of 22 °C or 55 °C. The obtained results are presented in Table 5.

TABLE 5
Sweet mixed-gas permeability and selectivity coefficients of BCARDO-
containing copolymers at various feed pressures and 22 °C or 55 °C.

| | | | Permeability | | | Selectivity | |
|---|---|---|---|---|---|---|---|
| | T | P | coefficients (Barrer) | | | coefficients | |
| Polymer | (°C) | (psi) | N₂ | CH₄ | CO₂ | N₂/CH₄ | CO₂/CH₄ |
| 6FDA-mPDA/BCARDO | 55 | 500 | 1.07 | 0.945 | 30.2 | 1.13 | 32.0 |
| (1:1) | | 700 | 1.01 | 0.942 | 30.2 | 1.07 | 32.1 |
| | | 900 | 0.957 | 0.947 | 30.5 | 1.01 | 32.2 |
| 6FDA-DAM/BCARDO | 22 | 300 | 1.76 | 2.51 | 93.1 | 0.701 | 37.1 |
| (1:1) | | 500 | 1.97 | 2.50 | 88.2 | 0.789 | 35.3 |
| | | 700 | 2.04 | 2.64 | 85.1 | 0.773 | 32.2 |
| | | 900 | 2.03 | 2.66 | 84.8 | 0.760 | 31.8 |
| 6FDA-DAM/BCARDO | 55 | 300 | — | — | — | — | — |
| (1:2) | | 500 | 3.06 | 2.87 | 82.7 | 1.07 | 28.8 |
| | | 700 | 3.13 | 3.05 | 81.0 | 1.02 | 26.5 |
| | | 900 | 3.41 | 3.17 | 80.1 | 1.07 | 25.3 |

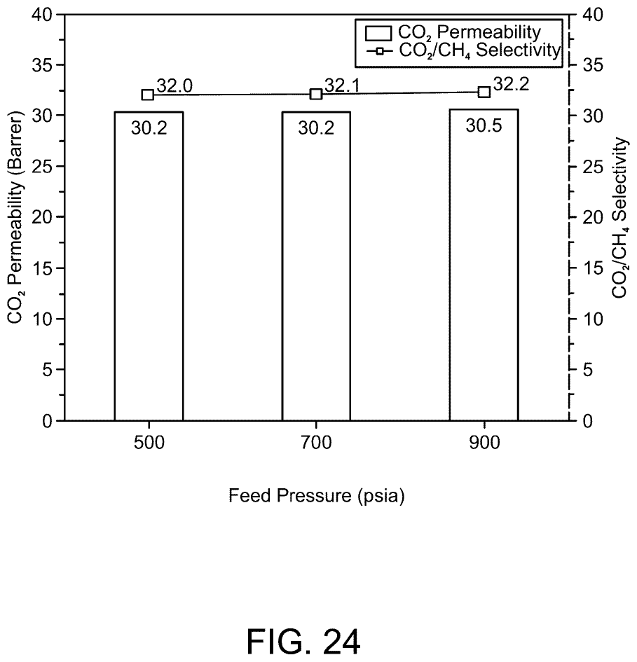

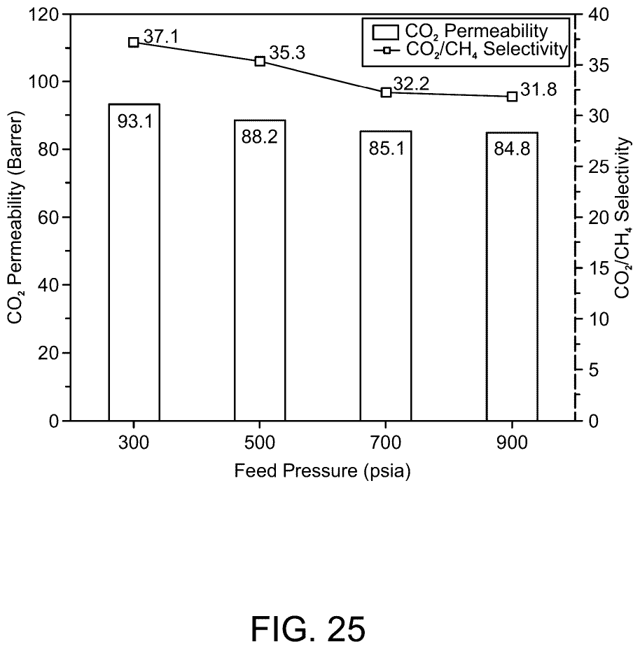

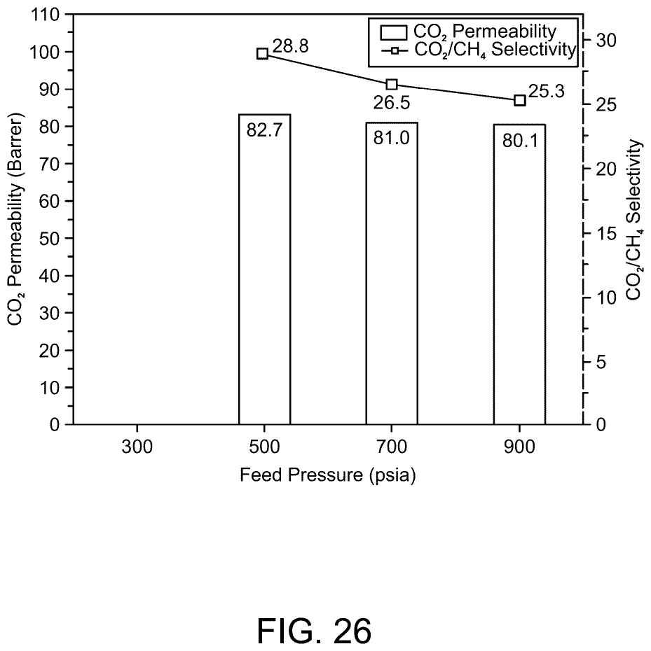

The mixed-gas CO₂ permeability coefficients and their corresponding CO₂/CH₄ selectivity values at various feed pressures measured in the present disclosure are illustrated in FIGS. 24-26. FIG. 24 is a plot showing a comparison between sweet mixed-gas CO₂ permeability (columns) and CO₂/CH₄ selectivity (curves) coefficients of 6FDA-mPDA/BCARDO (1:1) at different feed pressures ranging from 500 to 900 psi and 55 °C. FIG. 25 is a plot showing a comparison between sweet mixed-gas CO₂ permeability (columns) and CO₂/CH₄ selectivity (curves) coefficients of 6FDA-DAM/BCARDO (1:1) at different feed pressures ranging from 500 to 900 psi and 22 °C. FIG. 26 is a plot showing a comparison between sweet mixed-gas CO₂ permeability (columns) and CO₂/CH₄ selectivity (curves) coefficients of 6FDA-DAM/BCARDO (1:2) at different feed pressures ranging from 500 to 900 psi and 55 °C.

A general trend of decreasing CO₂ permeability with increasing feed pressure (300 to 900 psi) was observed. For instance, the CO₂ permeability of the 6FDA-DAM/BCARDO (1:1) membrane decreased by approximately 9% as the feed pressure increased from 300 psi to 900 psi. This reduction in permeability is attributed to competitive sorption among the gas components—specifically, competition for Langmuir sorption sites between CO₂ and other gases present in the mixture, such as N₂ and CH₄.

As a result of the decrease in the mixed-gas CO₂ permeability coefficients with slight changes in mixed-gas CH₄ permeability coefficients, the CO₂/CH₄ selectivity coefficients decreased when the pressure increased from 300 to 900 psi for all copolyimides as depicted in FIGS. 24-26. It can be noted that the mixed-gas CO₂/CH₄ selectivity showed a clear dependence of the type and molar ratio of comonomers. For instance, the higher the molar ratio of BCARDO within the polymer's backbone, the higher the mixed-gas CO₂/CH₄ selectivity coefficients.

### Temperature Effect

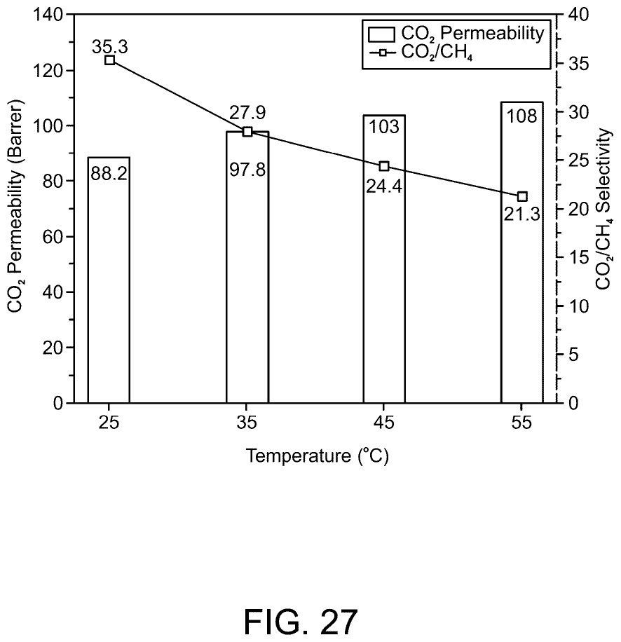

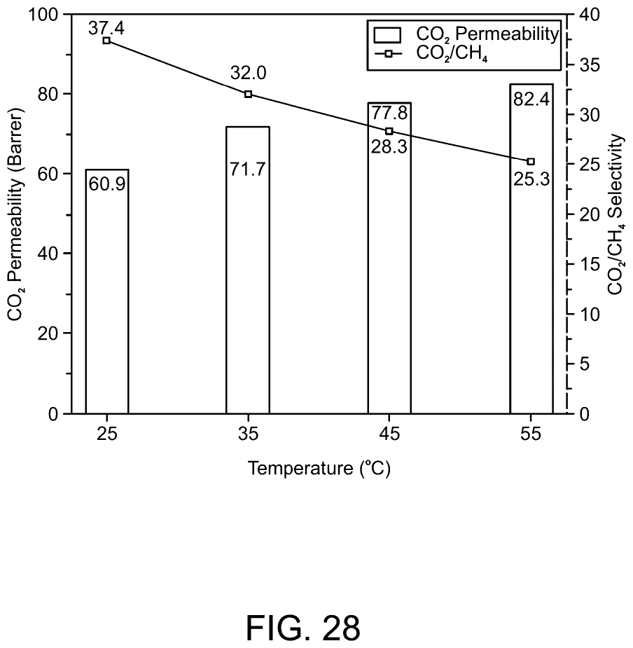

Based on the experimental results obtained from the gas feed pressure change on mixed-gas separation properties, the performance of the polymeric membranes were evaluated using the sweet gas mixture comprising 29.9 mol % CO₂, 60 mol % CH₄, and 10.1 mol % N₂, at different operating temperatures (25 °C, 35 °C, 45 °C, and 55 °C), under constant feed pressures of 500 psi or 900 psi. Representative results for the DAM:BCARDO series of copolyimide membranes are presented in FIGS. 27-28. FIG. 27 is a plot showing a comparison between sweet mixed-gas CO₂ permeability (columns) and CO₂/CH₄ selectivity (curves) coefficients of 6FDA-DAM/BCARDO (1:1) at different temperatures and a feed pressure of 500 psi. FIG. 28 is a plot showing a comparison between sweet mixed-gas CO₂ permeability (columns) and CO₂/CH₄ selectivity (curves) coefficients of 6FDA-DAM/BCARDO (1:2) at different temperatures and a feed pressure of 900 psi.

As shown in FIGS. 27-28, the mixed-gas CO₂ permeability coefficient increases with increasing operational temperature. This change can be attributed to the increase in kinetic energy of gaseous molecules in the mixture and the change in the dynamic free volume of polymeric chains within the membrane matrix. As a result, the mixed-gas CO₂/CH₄ selectivity coefficients tend to decrease with increasing temperature, which is in line with the known “trade-off” relationship between permeability and selectivity. The change in the selectivity coefficient is due to the increase in dynamic free volume of the membrane matrix, which results in allowing the slow diffusing molecules, such as methane, to diffuse faster, and hence, their corresponding permeability increases.

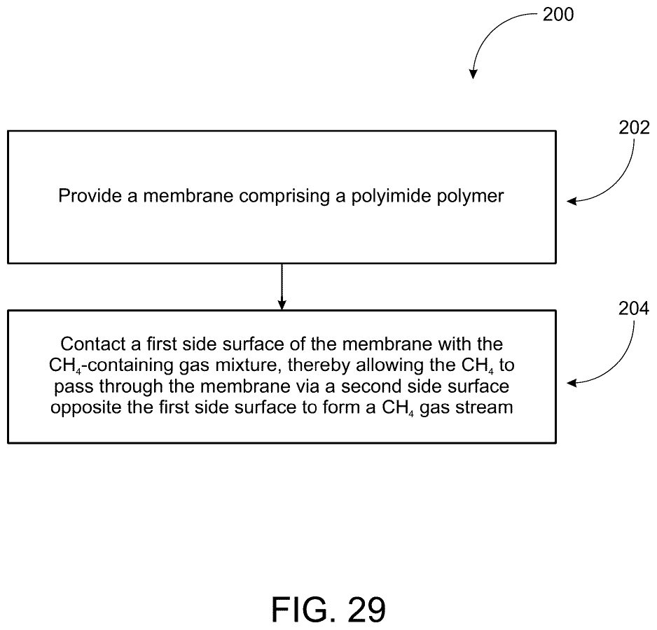

FIG. 29 is a method 200 for separating CH₄ from a CH₄-containing gas mixture. The method 200 begins at block 202 with the formation of a membrane comprising a polyimide polymer. At block 204, the method includes contacting a first side surface of the membrane with the CH₄-containing gas mixture, thereby allowing the CH₄ to pass through the membrane via a second side surface opposite the first side surface to form a CH₄ gas stream.

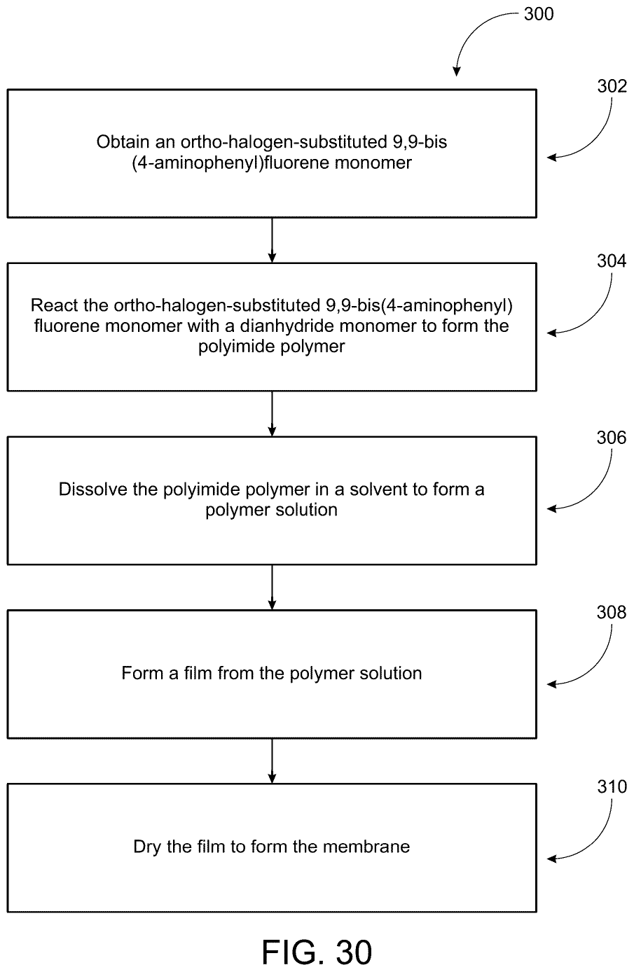

FIG. 30 is a method 300 for preparing the membrane comprising the polyimide polymer. The method 300 begins at block 302 with the synthesis or purchase of the monomers, such as an ortho-halogen-substituted 9,9-bis(4-aminophenyl)fluorene monomer. This may be performed by the techniques described herein. At block 304, the method 300 includes reacting the ortho-halogen-substituted 9,9-bis(4-aminophenyl)fluorene monomer with a dianhydride monomer to form the polyimide polymer. At block 306, the method includes dissolving the polyimide polymer in a solvent to form a polymer solution. At block 308, the method includes forming a film from the polymer solution. A film is formed from the polymer solution, for example, using the solvent evaporation techniques described herein. At block 310, the method includes drying the film to form the membrane. In some embodiments, the membrane is used to separate gas mixtures, for example, to sweeten natural gas by the separation of acid gases, such as H₂S, CO₂, or COS.

The present disclosure enables the development of polymeric materials exhibiting relatively high gas permeability and selectivity coefficients, thereby facilitating the fabrication of membrane modules with enhanced productivity and efficiency. The implementation of such high-performance membranes can reduce the cost of purifying natural gas reserves. In addition, these materials demonstrate improved chemical, thermal, physical, and mechanical stability, enabling them to withstand the harsh operating conditions typically encountered in gas separation processes.

While this specification contains many specific implementation details, these should not be construed as limitations on the scope of what may be claimed, but rather as descriptions of features that may be specific to particular implementations. Certain features that are described in this specification in the context of separate implementations can also be implemented, in combination, in a single implementation. Conversely, various features that are described in the context of a single implementation can also be implemented in multiple implementations, separately, or in any sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

### DEFINITIONS

When describing the present disclosure, the terms used are to be construed in accordance with the following definitions, unless a context dictates otherwise. Embodiments of the present invention will now be described more fully hereinafter with reference to the accompanying drawings wherever applicable, in that some, but not all embodiments of the disclosure are shown.

Unless otherwise defined, all technical and scientific terms used in this document have the same meaning as commonly understood by one of ordinary skill in the art to which the present application belongs. Methods and materials are described in this document for use in the present application; other, suitable methods and materials known in the art can also be used. The materials, methods, and examples are illustrative only and not intended to be limiting.

In the drawings, like reference numerals designate identical or corresponding parts throughout the several views. As used in this disclosure, the terms “a,” “an,” and “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed in this disclosure, and not otherwise defined, is for the purpose of description only and not of limitation. Any use of section headings is intended to aid reading of the document and is not to be interpreted as limiting; information that is relevant to a section heading may occur within or outside of that particular section.

Values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (for example, 1%, 2%, 3%, and 4%) and the sub-ranges (for example, 0.1% to 0.5%, 1.1% to 2.2%, and 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

The term “about,” as used in this disclosure, can allow for a degree of variability in a value or range, for example, within 10%, within 5%, or within 1% of a stated value or of a stated limit of a range.

As used herein, the terms “room temperature” and “ambient temperature” refer to a temperature in a range of 25 degrees Celsius (°C)±3 °C.

A weight percent of a component, unless specifically stated to the contrary, is based on the total weight of the formulation or composition in which the component is included. For example, if a particular element or component in a composition or article is said to have 5 wt. %, it is understood that this percentage is in relation to a total compositional percentage of 100%.

In the methods described in this disclosure, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The terms “sour” or “sour gas” mean that the gas stream contains high levels of hydrogen sulfide (H₂S).

The terms “sweet” or “sweet gas” mean that the gas stream contains low levels of hydrogen sulfide (H₂S).

As used in the present disclosure, the term “monomer unit,” used in reference to a polymer, refers to a monomer, or residue of a monomer, that has been incorporated into at least a portion of the polymer.

As used in the present disclosure, the term “polymerization product,” used in reference to one or more monomers, refers to a polymer that can be formed by a chemical reaction of the one or more monomers. For example, a “polymerization product” of acrylic acid is a polymer containing acrylic acid monomer units.

As used in the present disclosure, the term “Cₙ₋ₘ alkyl” refers to any linear or branched saturated hydrocarbon group having n to m carbons. Alkyl groups include, but are not limited to, methyl, ethyl, propyl such as propan-1-yl, propan-2-yl (iso-propyl), butyl such as butan-1-yl, butan-2-yl (sec-butyl), 2-methyl-propan-1-yl (iso-butyl), 2-methyl-propan-2-yl (t-butyl), pentyl, hexyl, octyl, decyl, and the like. As used in the present disclosure, the term “alkylene” refers to a bivalent alkyl.

As used in the present disclosure, the term “Cₙ₋ₘ haloalkyl” refers to the alkyl group defined above replaced by one or more halogens.

As used in the present disclosure, the term “Cₙ₋ₘ cycloalkyl” refers to a monocyclic or polycyclic non-aromatic radical, in which each of the ring-forming atoms (i.e., skeletal atoms) is a carbon atom. In one embodiment, the cycloalkyl group is saturated or partially unsaturated. In another embodiment, the cycloalkyl group is fused to an aromatic ring. Cycloalkyl groups include groups having 3 to 20 ring atoms.

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term “cycloalkyl” includes “unsaturated non-aromatic carbocyclyl” and “non-aromatic unsaturated carbocyclyl” groups, which refer to a non-aromatic carbocycle as defined herein, containing at least one carbon double bond or one carbon triple bond.

As used herein, the term “heterocycloalkyl,” “heterocyclyl,” or “heterocyclylic” refers to an aliphatic heterocyclyl containing one to four ring heteroatoms, each selected from O, S, and N. In one embodiment, each heterocycloalkyl group has from 4 to 20 atoms in their ring system, provided that the ring of that group does not contain two adjacent O or S atoms. In another embodiment, the heterocycloalkyl group is fused to an aromatic ring. In one embodiment, the nitrogen and sulfur heteroatoms can be optionally oxidized, and the nitrogen atom can be optionally quaternized. The heterocyclic system can be attached, unless otherwise noted, at any heteroatom or carbon atom that provides a stable structure. A heterocycle can be aromatic or non-aromatic in nature. In one embodiment, the heterocycle is a heteroaryl. An example of a 3-membered heterocycloalkyl group includes, and is not limited to, aziridine. Examples of 4-membered heterocycloalkyl groups include, and are not limited to, azetidine and a beta lactam. Examples of 5-membered heterocycloalkyl groups include, and are not limited to, pyrrolidine, oxazolidine, and thiazolidinedione. Examples of 6-membered heterocycloalkyl groups include, and are not limited to, piperidine, morpholine, and piperazine.

Examples of non-aromatic heterocycles include, but are not limited to, monocyclic groups such as aziridine, oxirane, thiirane, azetidine, oxetane, thiethane, pyrrolidine, pyrroline, pyrazolidine, imidazoline, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethylene oxide.

As used herein, the term “aromatic” refers to a carbocycle or heterocycle with one or more polyunsaturated rings and having an aromatic character, that is, having delocalized (4n 2) p (pi) electrons, where n is a whole number.

As used herein, the term “aryl,” used alone or in combination with other terms, means, unless otherwise noted, a carbocyclic aromatic system containing one or more rings (typically one, two or three rings), wherein said rings may be pendant together, such as a biphenyl, or they may be fused, such as naphthalene. Examples of aryl groups include, but are not limited to, phenyl, anthracyl, and naphthyl.

As used herein, the term “heteroaryl” and “heteroaromatic” refer to a heterocycle having aromatic character. A polycyclic heteroaryl can include one or more rings that are partially saturated. Examples of heteroaryl groups also include, but are not limited to, pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

As used in the present disclosure, the term “polycyclic aromatic hydrocarbon” refers to any multiple-condensed ring system of two or more fused, all-carbon aromatic rings. Polycyclic aromatic hydrocarbons include, but are not limited to, naphthalene, anthracene, triphenylene, pyrene, perylene, and the like. In some embodiments, the polycyclic aromatic hydrocarbon includes polycyclic heterocycles and heteroaryls. Examples of polycyclic heterocycles and heteroaryls include indolyl (particularly 3-, 4-, 5-, 6-, and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benzotriazolyl, thioxanthinyl, carbazolyl, carbolinyl, acridinyl, pyrrolizidinyl, and quinolizidinyl.

As used in the present disclosure, the term “halo” refers to —F, —Cl, —Br, or —I.

As used in the present disclosure, the term “hydroxyl” refers to —OH.

As used in the present disclosure, the term “amino” refers to —NH₂.

As used in the present disclosure, the term “thiol” refers to —SH.

As used in the present disclosure, the term “carboxyl” refers to —C(O)OH.

As used in the present disclosure, the term “azido” refers to —N₃.

Where a variable of the present disclosure defines a group having more than one substituent (for example, group A of Formula (I)) and the Markush group definition for that variable lists, for example, a polycyclic aromatic hydrocarbon, then it is understood that the polycyclic aromatic hydrocarbon represents a substituent having the necessary valency.

### Embodiments

Embodiments described in examples herein provide a method for separating methane (CH₄) from a CH₄-containing gas mixture, wherein the method comprises:
providing a membrane comprising a polyimide polymer comprising a monomer having a structure of Formula (I):

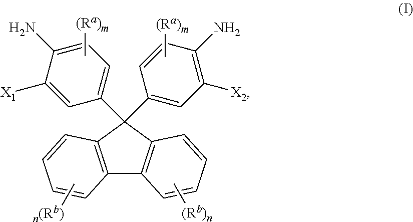

wherein:
   X₁ and X₂ are each independently selected F, Cl, Br, and I;
   R^{a} and R^{b} are each independently selected from H, D, CN, halo, C₁₋₆ alkyl, C₂₋₆ alkenyl, C₂₋₆ alkynyl, C₁₋₆ haloalkyl, C₁₋₆ alkoxy, C₁₋₆ haloalkoxy, C₆₋₁₀ aryl, C₃₋₁₄ cycloalkyl, 5-14 membered heteroaryl, 4-14 membered heterocycloalkyl, C₆₋₁₀ aryl-C₁₋₄ alkyl-, C₃₋₁₄ cycloalkyl-C₁₋₄ alkyl-, (5-14 membered heteroaryl)-C₁₋₄ alkyl-, (4-14 membered heterocycloalkyl)-C₁₋₄ alkyl-, NR^{c}R^{c}, NR^{c}C(O)R^{c}, and NR^{c}C(O)OR^{c}, wherein the C₁₋₆ alkyl, C₂₋₆ alkenyl, C₂₋₆ alkynyl, C₁₋₆ haloalkyl, C₁₋₆ alkoxy, C₁₋₆ haloalkoxy, C₆₋₁₀ aryl, C₃₋₁₄ cycloalkyl, 5-14 membered heteroaryl, 4-14 membered heterocycloalkyl, C₆₋₁₀ aryl-C₁₋₄ alkyl-, C₃₋₁₄ cycloalkyl-C₁₋₄ alkyl-, (5-14 membered heteroaryl)-C₁₋₄ alkyl-, and (4-14 membered heterocycloalkyl)-C₁₋₄ alkyl- of R^{a} and R^{b} are each optionally substituted with 1, 2, 3, 4 or 5 independently selected R^{c} substituents; or
   wherein R^{a} and R^{b} taken together with the C atom to which they are attached form a 6-14 membered aromatic group, or 6-14 membered heteroaromatic group, wherein the 6-14 membered aromatic group or the 6-14 membered heteroaromatic group are optionally substituted with 1, 2, 3, or 4 independently selected R^{c} substituents;
   each R^{c} is independently selected from H, D, halo, C₁₋₆ alkyl, C₁₋₆ alkoxy, CN, OH, oxo, NH₂, NHC₁₋₆ alkyl, and N(C₁₋₆ alkyl)₂;
   m is an integer selected from 0, 1, 2, and 3;
   n is an integer selected from 0, 1, 2, 3, and 4; and
contacting a first side surface of the membrane with the CH₄-containing gas mixture, thereby allowing the CH₄ to pass through the membrane via a second side surface opposite the first side surface to form a CH₄ gas stream.

In an aspect, the CH₄-containing gas mixture further comprises one or more components selected the group consisting of carbon dioxide (CO₂), hydrogen (H₂), helium (He), and nitrogen (N₂).

In an aspect combinable with any of the previous aspects, the CH₄-containing gas mixture further comprises hydrogen sulfide (H₂S).

In an aspect combinable with any of the previous aspects, the polyimide polymer of the membrane comprises a copolymer having a structure of Formula (II):

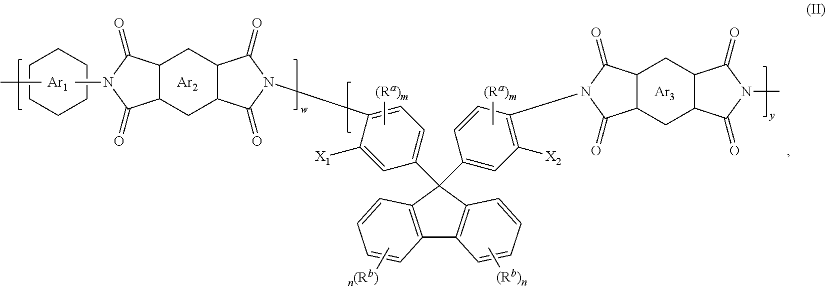

wherein Ar₁, Ar₂, and Ar₃ independently comprise aromatic moieties, bridging moieties, or both.

In an aspect combinable with any of the previous aspects, a ratio of w to y is between 3:1 and 1:3.

In an aspect combinable with any of the previous aspects, the polyimide polymer comprises a random copolymer.

In an aspect combinable with any of the previous aspects, the polyimide polymer comprises a block copolymer.

In an aspect combinable with any of the previous aspects, the polyimide polymer of the membrane comprises a homopolymer having a structure of Formula (III):

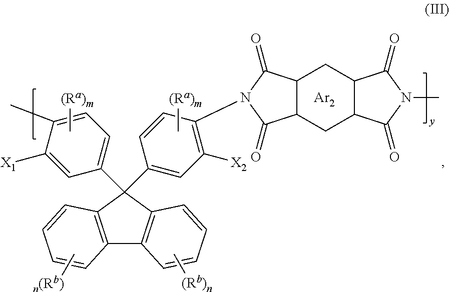

wherein Ar₁ comprises an aromatic moiety or a bridging moiety.

In an aspect combinable with any of the previous aspects, the polyimide polymer of the membrane comprises a copolymer having a structure of Formula (III-A), (III-A), or (III-C):

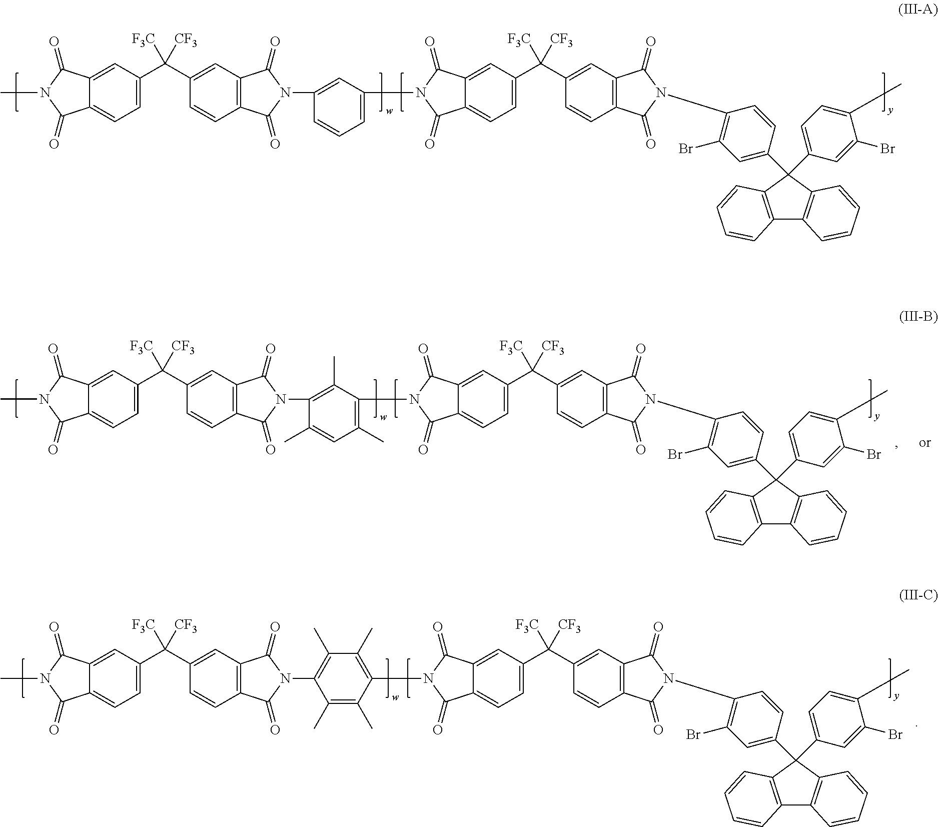

In an aspect combinable with any of the previous aspects, a ratio of w to y is between 3:1 and 1:3.

In an aspect combinable with any of the previous aspects, the polyimide polymer comprises a random copolymer.

In an aspect combinable with any of the previous aspects, the polyimide polymer comprises a block copolymer.

In an aspect combinable with any of the previous aspects, the polyimide polymer of the membrane comprises a homopolymer having a structure of Formula (IV):

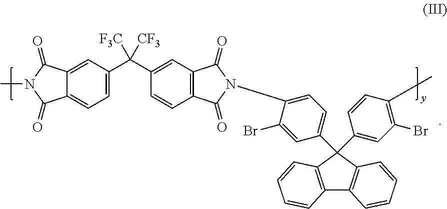

In an aspect combinable with any of the previous aspects, the polyimide polymer further comprises a diamine monomer of Formula (V):

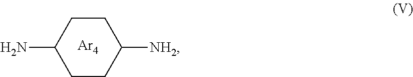

wherein Ar₄ comprises an aromatic moiety or a bridging moiety.

In an aspect combinable with any of the previous aspects, the diamine monomer is selected from the group consisting of

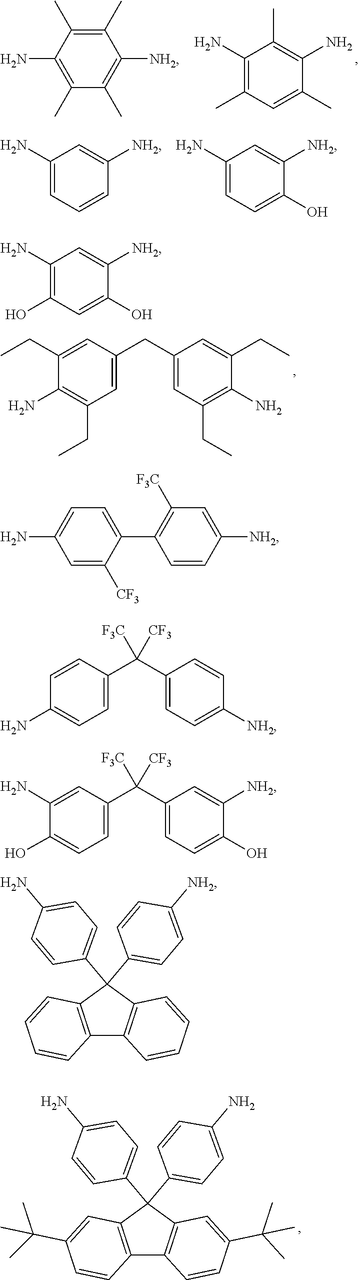

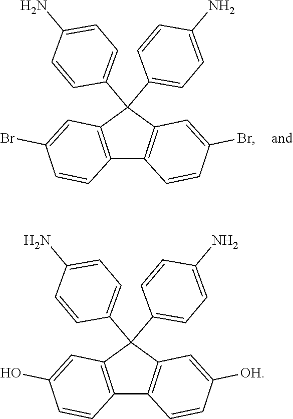 [SMILES: Nc1ccc(C2(c3ccc(N)cc3)c3cc(Br)ccc3-c3ccc(Br)cc32)cc1 ; Nc1ccc(C2(c3ccc(N)cc3)c3cc(O)ccc3-c3ccc(O)cc32)cc1]

In an aspect combinable with any of the previous aspects, the polyimide polymer further comprises a dianhydride monomer selected from the group consisting of

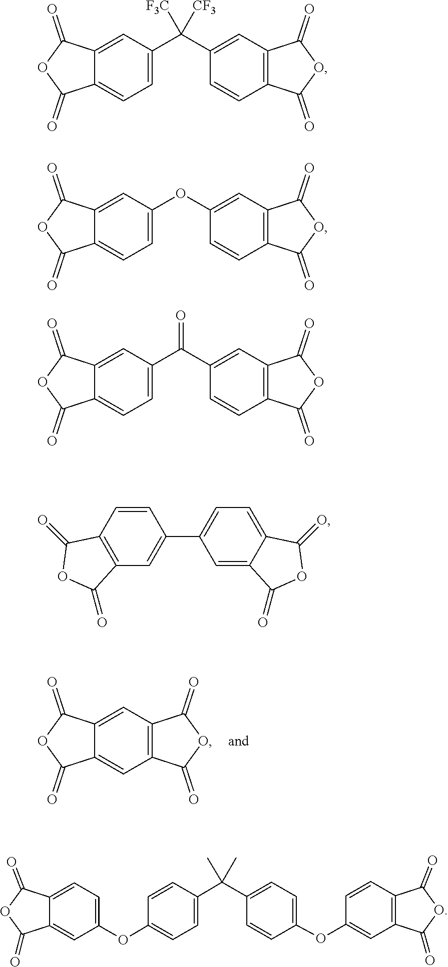

In an aspect combinable with any of the previous aspects, the method further comprises preparing the membrane comprising the polyimide polymer by: obtaining an ortho-halogen-substituted 9,9-bis(4-aminophenyl)fluorene monomer of Formula (I); reacting the ortho-halogen-substituted 9,9-bis(4-aminophenyl)fluorene monomer with a dianhydride monomer to form the polyimide polymer; dissolving the polyimide polymer in a solvent to form a polymer solution; forming a film from the polymer solution; and drying the film to form the membrane.

In an aspect combinable with any of the previous aspects, the ortho-halogen-substituted 9,9-bis(4-aminophenyl)fluorene monomer is formed by reacting a substituted or unsubstituted 9,9-bis(4-aminophenyl)fluorene monomer with a halogenating agent.

In an aspect combinable with any of the previous aspects, the ortho-halogen-substituted 9,9-bis(4-aminophenyl)fluorene monomer is selected from the group consisting of

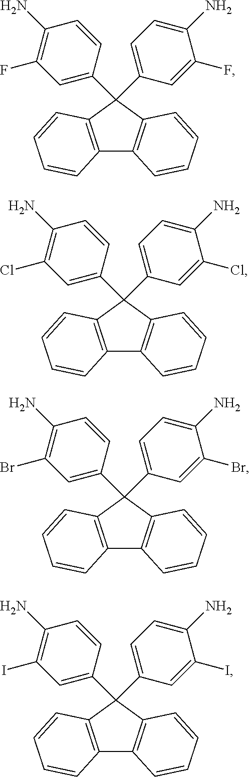

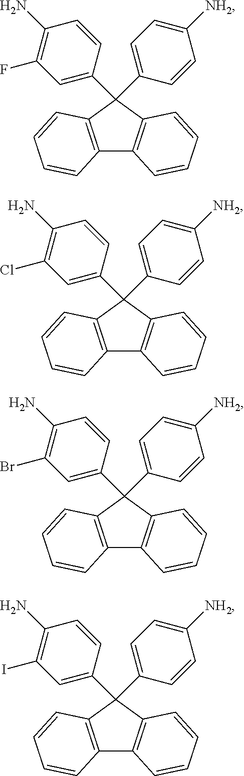

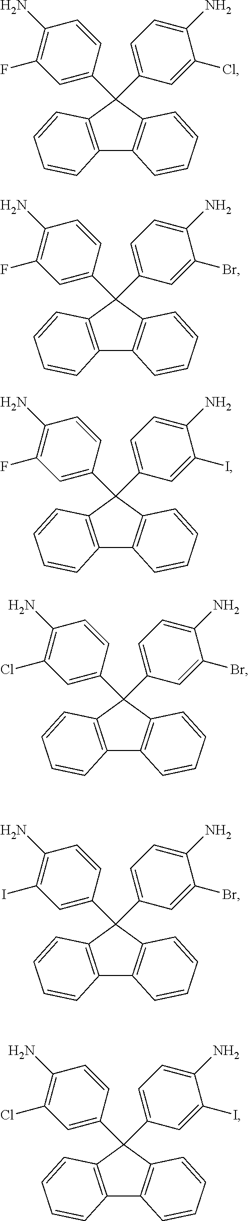

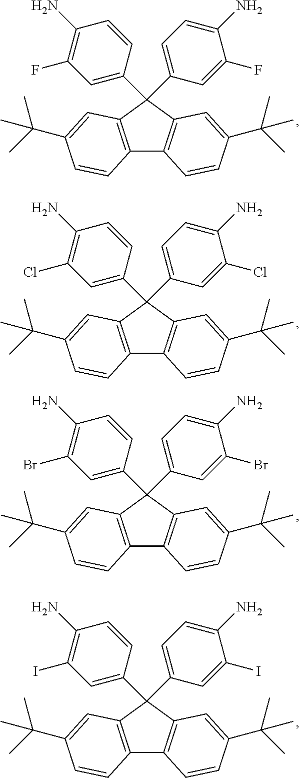

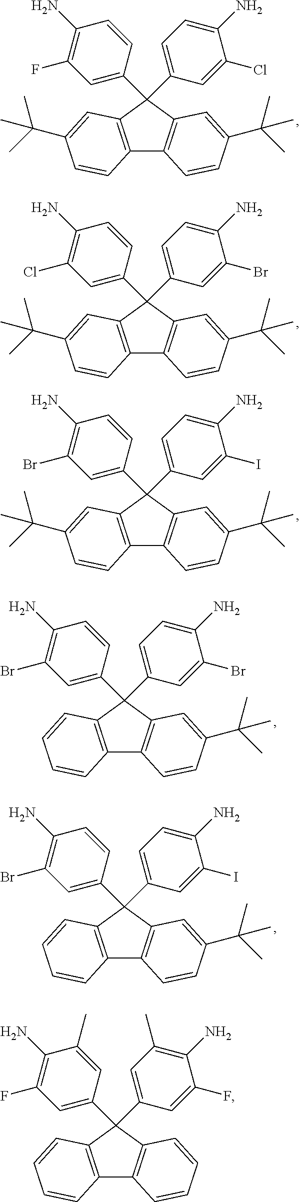

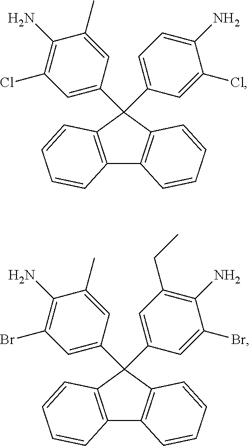 [SMILES: Cc1cc(C2(c3ccc(N)c(Cl)c3)c3ccccc3-c3ccccc32)cc(Cl)c1N ; CCc1cc(C2(c3cc(C)c(N)c(Br)c3)c3ccccc3-c3ccccc32)cc(Br)c1N]

or any combinations thereof.

In an aspect combinable with any of the previous aspects, the polyimide polymer is formed by heating a mixture of ortho-halogen-substituted 9,9-bis(4-aminophenyl)fluorene monomer, the dianhydride monomer, and a diamine monomer.

Embodiments described in examples herein also provide a membrane comprising a polyimide polymer comprising a monomer having a structure of Formula (I):

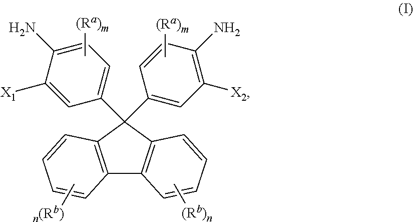

wherein:
X₁ and X₂ are each independently selected F, Cl, Br, and I;
R^{a} and R^{b} are each independently selected from H, D, CN, halo, C₁₋₆ alkyl, C₂₋₆ alkenyl, C₂₋₆ alkynyl, C₁₋₆ haloalkyl, C₁₋₆ alkoxy, C₁₋₆ haloalkoxy, C₆₋₁₀ aryl, C₃₋₁₄ cycloalkyl, 5-14 membered heteroaryl, 4-14 membered heterocycloalkyl, C₆₋₁₀ aryl-C₁₋₄ alkyl-, C₃₋₁₄ cycloalkyl-C₁₋₄ alkyl-, (5-14 membered heteroaryl)-C₁₋₄ alkyl-, (4-14 membered heterocycloalkyl)-C₁₋₄ alkyl-, NR^{c}R^{c}, NR^{c}C(O)R^{c}, and NR^{c}C(O)OR^{c}, wherein the C₁₋₆ alkyl, C₂₋₆ alkenyl, C₂₋₆ alkynyl, C₁₋₆ haloalkyl, C₁₋₆ alkoxy, C₁₋₆ haloalkoxy, C₆₋₁₀ aryl, C₃₋₁₄ cycloalkyl, 5-14 membered heteroaryl, 4-14 membered heterocycloalkyl, C₆₋₁₀ aryl-C₁₋₄ alkyl-, C₃₋₁₄ Cycloalkyl-C₁₋₄ alkyl-, (5-14 membered heteroaryl)-C₁₋₄ alkyl-, and (4-14 membered heterocycloalkyl)-C₁₋₄ alkyl- of R^{a} and R^{b} are each optionally substituted with 1, 2, 3, 4 or 5 independently selected Re substituents; or
wherein R^{a} and R^{b} taken together with the C atom to which they are attached form a 6-14 membered aromatic group, or 6-14 membered heteroaromatic group, wherein the 6-14 membered aromatic group or the 6-14 membered heteroaromatic group are optionally substituted with 1, 2, 3, or 4 independently selected Re substituents;
each R^{c} is independently selected from H, D, halo, C₁₋₆ alkyl, C₁₋₆ alkoxy, CN, OH, oxo, NH₂, NHC₁₋₆ alkyl, and N(C₁₋₆ alkyl)₂;
m is an integer selected from 0, 1, 2, and 3;
n is an integer selected from 0, 1, 2, 3, and 4; and
the membrane has a CO₂/CH₄ selectivity of from about 20 to about 40 at a temperature of 25 to 55 °C and a pressure of 200 to 1200 psi.

In an aspect combinable with any of the previous aspects, the polyimide polymer of the membrane comprises a copolymer having a structure of Formula (II):

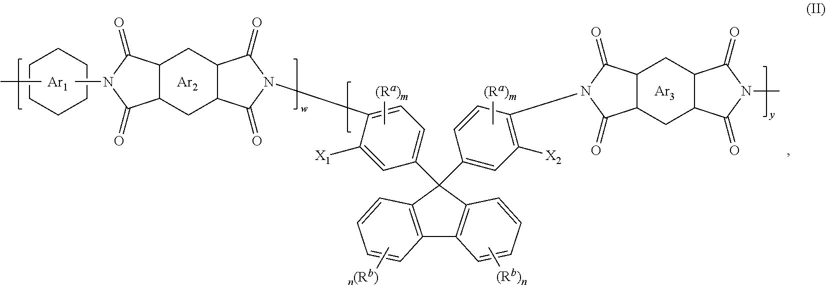

wherein Ar₁, Ar₂, and Ar₃ independently comprise aromatic moieties, bridging moieties, or both.

In an aspect combinable with any of the previous aspects, the polyimide polymer of the membrane comprises a homopolymer having a structure of Formula (III):

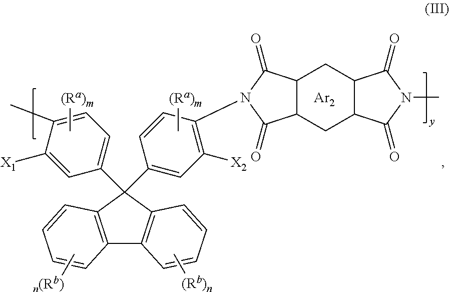

wherein Ar₁ comprises an aromatic moiety or a bridging moiety.

In an aspect combinable with any of the previous aspects, the polyimide polymer of the membrane comprises a copolymer having a structure of Formula (III-A), (III-A), or (III-C):

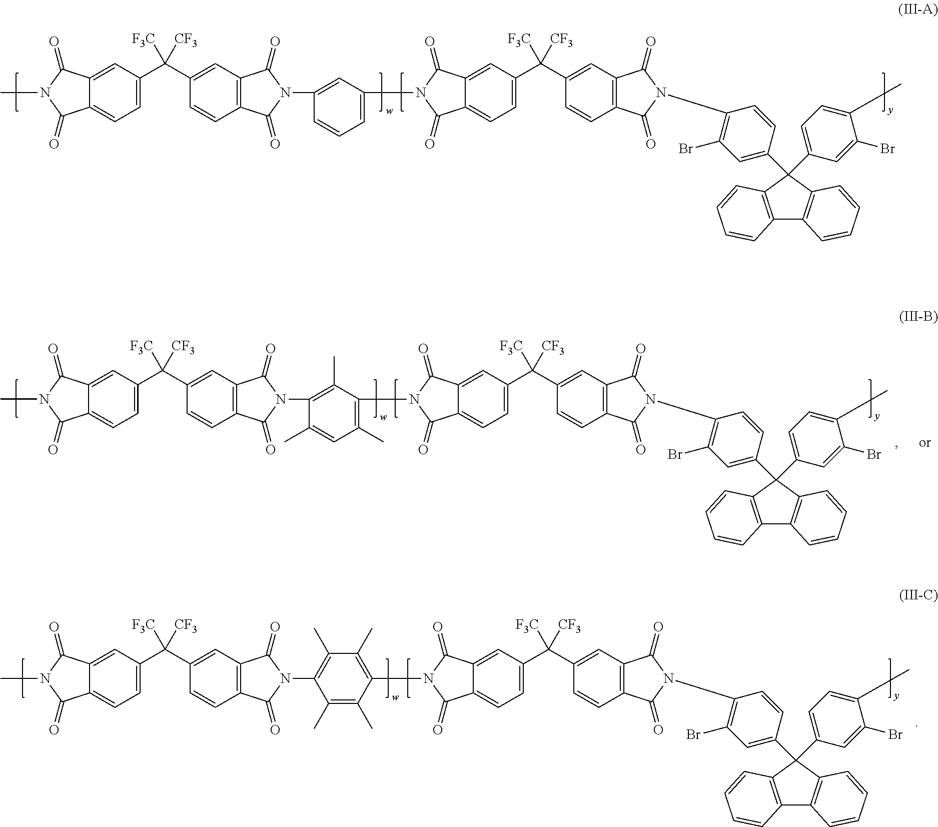

In an aspect combinable with any of the previous aspects, the polyimide polymer of the membrane comprises a homopolymer having a structure of Formula (IV):

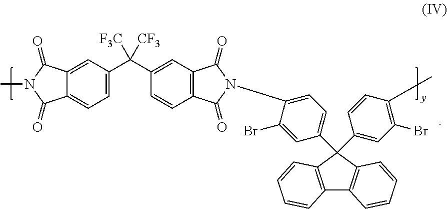

Other implementations are also within the scope of the following claims.

What is claimed is:

1. A method for separating methane (CH₄) from a CH₄-containing gas mixture, wherein the method comprises:
providing a membrane comprising a polyimide polymer comprising a monomer having a structure of Formula (I): 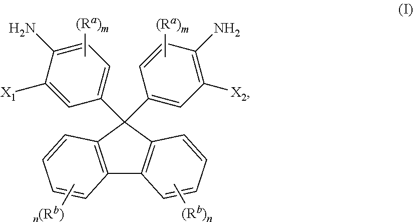
wherein:
X₁ and X₂ are each independently selected F, Cl, Br, and I;
R^{a} and R^{b} are each independently selected from H, D, CN, halo, C₁₋₆ alkyl, C₂₋₆ alkenyl, C₂₋₆ alkynyl, C₁₋₆ haloalkyl, C₁₋₆ alkoxy, C₁₋₆ haloalkoxy, C₆₋₁₀ aryl, C₃₋₁₄ cycloalkyl, 5-14 membered heteroaryl, 4-14 membered heterocycloalkyl, C₆₋₁₀ aryl-C₁₋₄ alkyl-, C₃₋₁₄ cycloalkyl-C₁₋₄ alkyl-, (5-14 membered heteroaryl)-C₁₋₄ alkyl-, (4-14 membered heterocycloalkyl)-C₁₋₄ alkyl-, NR^{c}R^{c}, NR^{c}C(O)R^{c}, and NR^{c}C(O)OR^{c}, wherein the C₁₋₆ alkyl, C₂₋₆ alkenyl, C₂₋₆ alkynyl, C₁₋₆ haloalkyl, C₁₋₆ alkoxy, C₁₋₆ haloalkoxy, C₆₋₁₀ aryl, C₃₋₁₄ cycloalkyl, 5-14 membered heteroaryl, 4-14 membered heterocycloalkyl, C₆₋₁₀ aryl-C₁₋₄ alkyl-, C₃₋₁₄ cycloalkyl-C₁₋₄ alkyl-, (5-14 membered heteroaryl)-C₁₋₄ alkyl-, and (4-14 membered heterocycloalkyl)-C₁₋₄ alkyl- of R^{a} and R^{b} are each optionally substituted with 1, 2, 3, 4 or 5 independently selected R^{c} substituents; or
wherein R^{a} and R^{b} taken together with the C atom to which they are attached form a 6-14 membered aromatic group, or 6-14 membered heteroaromatic group, wherein the 6-14 membered aromatic group or the 6-14 membered heteroaromatic group are optionally substituted with 1, 2, 3, or 4 independently selected Re substituents;
each R^{c} is independently selected from H, D, halo, C₁₋₆ alkyl, C₁₋₆ alkoxy, CN, OH, oxo, NH₂, NHC₁₋₆ alkyl, and N(C₁₋₆ alkyl)₂;
m is an integer selected from 0, 1, 2, and 3;
n is an integer selected from 0, 1, 2, 3, and 4; and
contacting a first side surface of the membrane with the CH₄-containing gas mixture, thereby allowing the CH₄ to pass through the membrane via a second side surface opposite the first side surface to form a CH₄ gas stream.

2. The method of claim 1, wherein the CH₄-containing gas mixture further comprises one or more components selected the group consisting of carbon dioxide (CO₂), hydrogen (H₂), helium (He), and nitrogen (N₂).

3. The method of claim 2, wherein the CH₄-containing gas mixture further comprises hydrogen sulfide (H₂S).

4. The method of claim 1, wherein the polyimide polymer of the membrane comprises a copolymer having a structure of Formula (II): 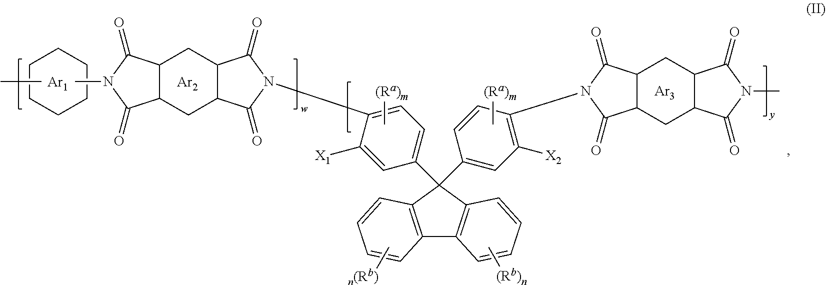
wherein Ar₁, Ar₂, and Ar₃ independently comprise aromatic moieties, bridging moieties, or both.

5. The method of claim 4, wherein a ratio of w to y is between 3:1 and 1:3.

6. The method of claim 4, wherein the polyimide polymer comprises a random copolymer.

7. The method of claim 4, wherein the polyimide polymer comprises a block copolymer.

8. The method of claim 1, wherein the polyimide polymer of the membrane comprises a homopolymer having a structure of Formula (III): 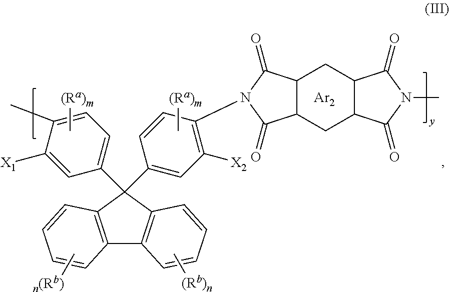
wherein Ar₁ comprises an aromatic moiety or a bridging moiety.

9. The method of claim 1, wherein the polyimide polymer of the membrane comprises a copolymer having a structure of Formula (III-A), (III-A), or (III-C):

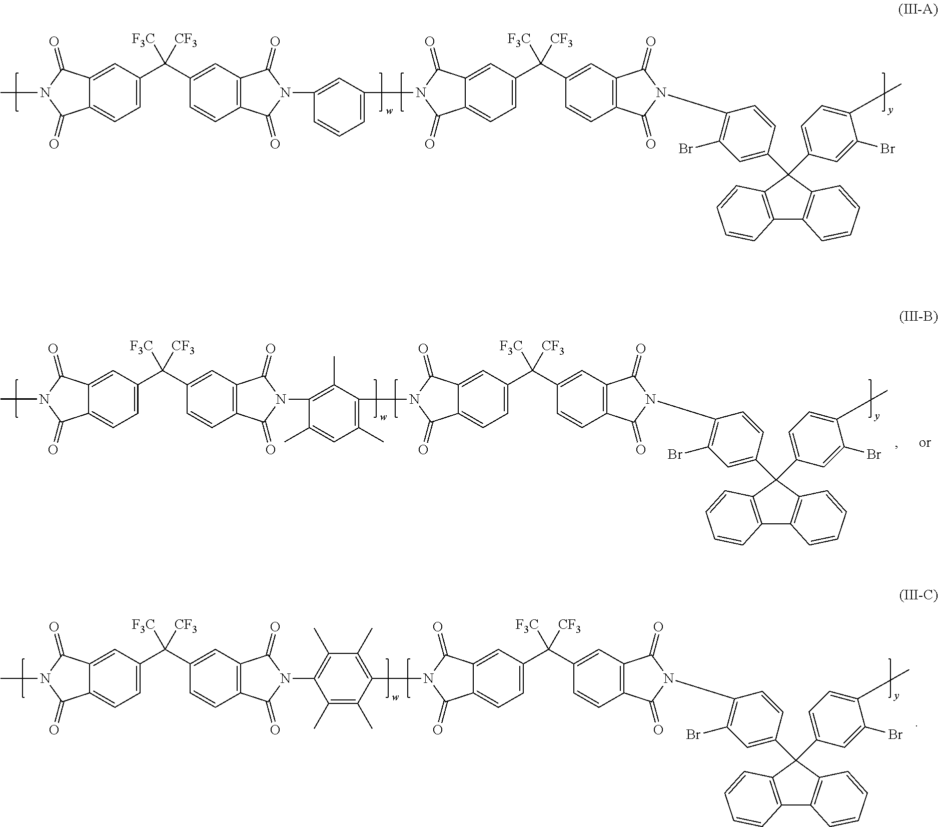

10. The method of claim 9, wherein a ratio of w to y is between 3:1 and 1:3.

11. The method of claim 9, wherein the polyimide polymer comprises a random copolymer.

12. The method of claim 9, wherein the polyimide polymer comprises a block copolymer.

13. The method of claim 1, wherein the polyimide polymer of the membrane comprises a homopolymer having a structure of Formula (IV):

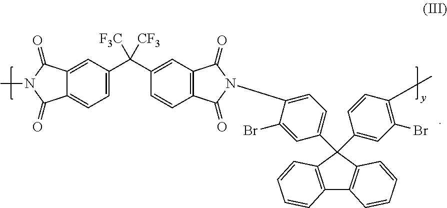

14. The method of claim 1, wherein the polyimide polymer further comprises a diamine monomer of Formula (V): 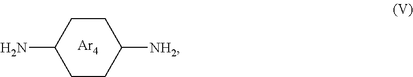
wherein Ar₄ comprises an aromatic moiety or a bridging moiety.

15. The method of claim 14, wherein the diamine monomer is selected from the group consisting of

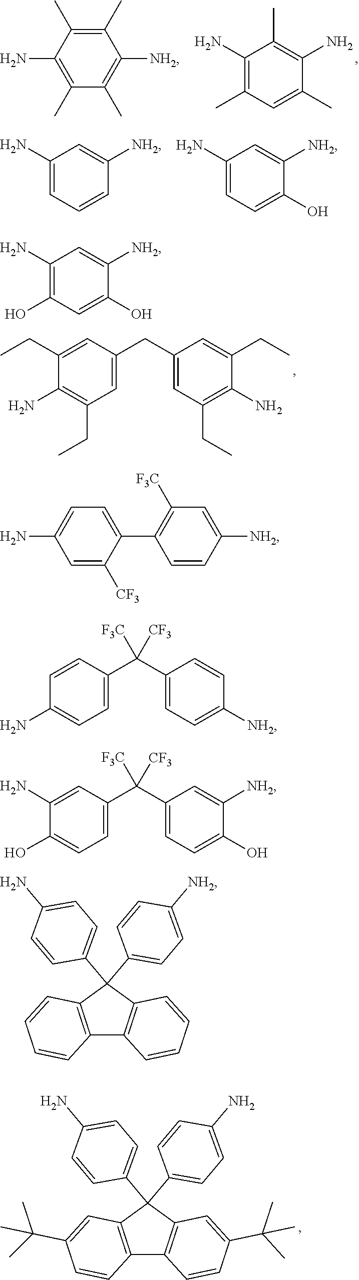

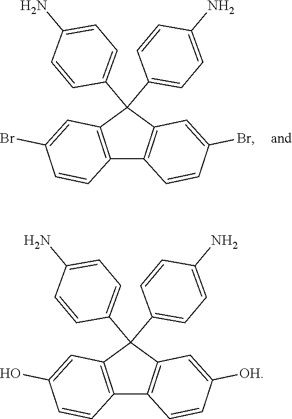 [SMILES: Nc1ccc(C2(c3ccc(N)cc3)c3cc(Br)ccc3-c3ccc(Br)cc32)cc1 ; Nc1ccc(C2(c3ccc(N)cc3)c3cc(O)ccc3-c3ccc(O)cc32)cc1]

16. The method of claim 1, wherein the polyimide polymer further comprises a dianhydride monomer selected from the group consisting of

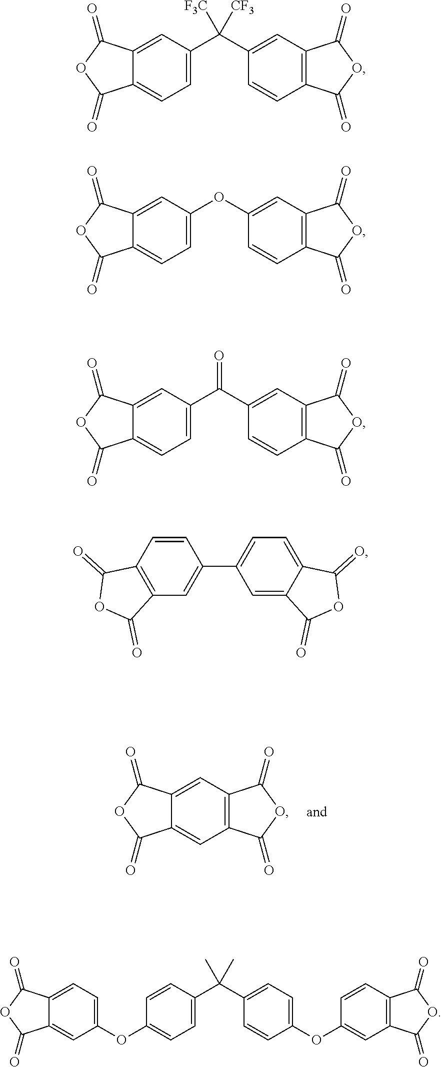

17. The method of claim 1, further comprising preparing the membrane comprising the polyimide polymer by:
obtaining an ortho-halogen-substituted 9,9-bis(4-aminophenyl)fluorene monomer of Formula (I);
reacting the ortho-halogen-substituted 9,9-bis(4-aminophenyl)fluorene monomer with a dianhydride monomer to form the polyimide polymer;
dissolving the polyimide polymer in a solvent to form a polymer solution;
forming a film from the polymer solution; and
drying the film to form the membrane.

18. The method of claim 17, wherein the ortho-halogen-substituted 9,9-bis(4-aminophenyl)fluorene monomer is formed by reacting a substituted or unsubstituted 9,9-bis(4-aminophenyl)fluorene monomer with a halogenating agent.

19. The method of claim 17, wherein the ortho-halogen-substituted 9,9-bis(4-aminophenyl)fluorene monomer is selected from the group consisting of 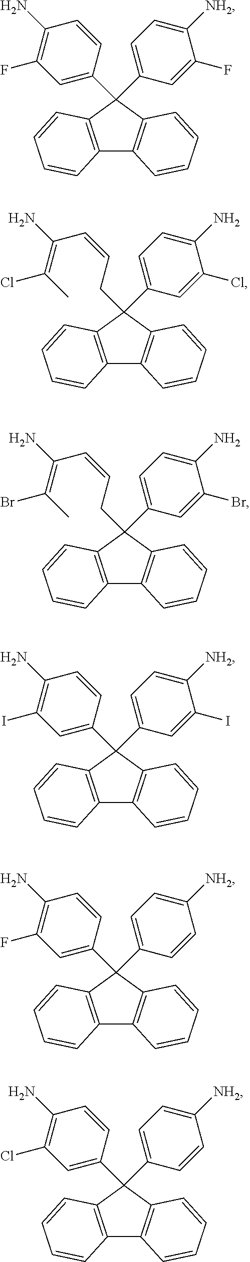 [SMILES: Nc1ccc(C2(c3ccc(N)c(F)c3)c3ccccc3-c3ccccc32)cc1F ; C/C(Cl)=C(N)\C=C/CC1(c2ccc(N)c(Cl)c2)c2ccccc2-c2ccccc21 ; C/C(Br)=C(N)\C=C/CC1(c2ccc(N)c(Br)c2)c2ccccc2-c2ccccc21 ; Nc1ccc(C2(c3ccc(N)c(I)c3)c3ccccc3-c3ccccc32)cc1I ; Nc1ccc(C2(c3ccc(N)c(F)c3)c3ccccc3-c3ccccc32)cc1 ; Nc1ccc(C2(c3ccc(N)c(Cl)c3)c3ccccc3-c3ccccc32)cc1] 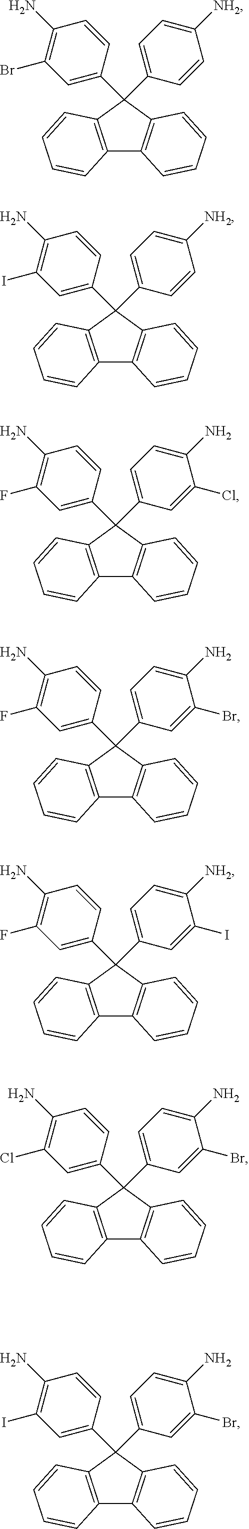 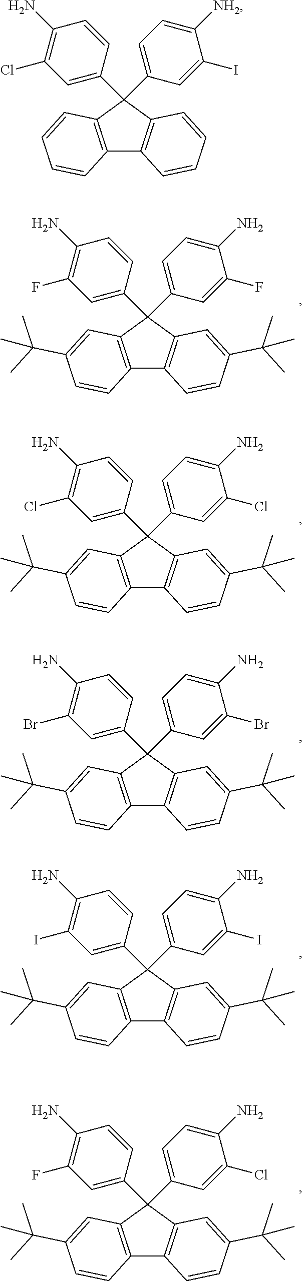 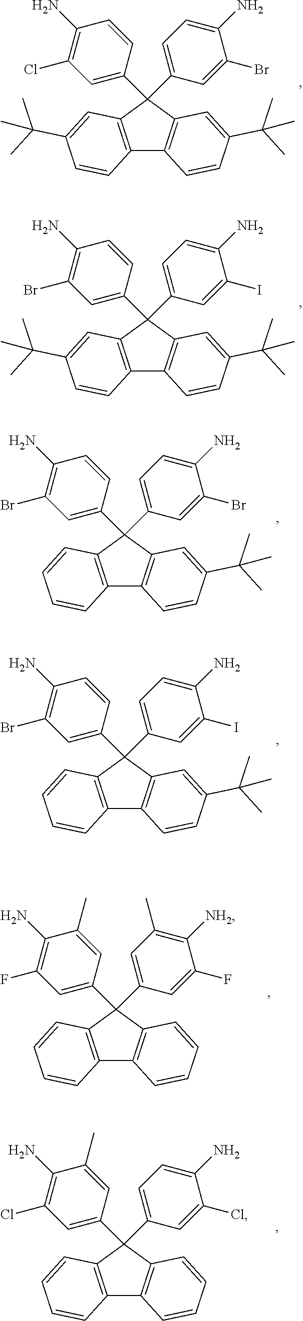 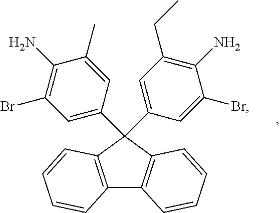 [SMILES: CCc1cc(C2(c3cc(C)c(N)c(Br)c3)c3ccccc3-c3ccccc32)cc(Br)c1N]
or any combinations thereof.

20. The method of claim 17, wherein the polyimide polymer is formed by heating a mixture of ortho-halogen-substituted 9,9-bis(4-aminophenyl)fluorene monomer, the dianhydride monomer, and a diamine monomer.

21. A membrane comprising a polyimide polymer comprising a monomer having a structure of Formula (I): 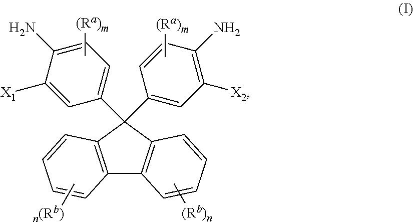
wherein:
X₁ and X₂ are each independently selected F, Cl, Br, and I;
R^{a} and R^{b} are each independently selected from H, D, CN, halo, C₁₋₆ alkyl, C₂₋₆ alkenyl, C₂₋₆ alkynyl, C₁₋₆ haloalkyl, C₁₋₆ alkoxy, C₁₋₆ haloalkoxy, C₆₋₁₀ aryl, C₃₋₁₄ cycloalkyl, 5-14 membered heteroaryl, 4-14 membered heterocycloalkyl, C₆₋₁₀ aryl-C₁₋₄ alkyl-, C₃₋₁₄ cycloalkyl-C₁₋₄ alkyl-, (5-14 membered heteroaryl)-C₁₋₄ alkyl-, (4-14 membered heterocycloalkyl)-C₁₋₄ alkyl-, NR^{c}R^{c}, NR^{c}C(O)R^{c}, and NR^{c}C(O)OR^{c}, wherein the C₁₋₆ alkyl, C₂₋₆ alkenyl, C₂₋₆ alkynyl, C₁₋₆ haloalkyl, C₁₋₆ alkoxy, C₁₋₆ haloalkoxy, C₆₋₁₀ aryl, C₃₋₁₄ cycloalkyl, 5-14 membered heteroaryl, 4-14 membered heterocycloalkyl, C₆₋₁₀ aryl-C₁₋₄ alkyl-, C₃₋₁₄ cycloalkyl-C₁₋₄ alkyl-, (5-14 membered heteroaryl)-C₁₋₄ alkyl-, and (4-14 membered heterocycloalkyl)-C₁₋₄ alkyl- of R^{a} and R^{b} are each optionally substituted with 1, 2, 3, 4 or 5 independently selected R^{c} substituents; or
wherein R^{a} and R^{b} taken together with the C atom to which they are attached form a 6-14 membered aromatic group, or 6-14 membered heteroaromatic group, wherein the 6-14 membered aromatic group or the 6-14 membered heteroaromatic group are optionally substituted with 1, 2, 3, or 4 independently selected Re substituents;
each R^{c} is independently selected from H, D, halo, C₁₋₆ alkyl, C₁₋₆ alkoxy, CN, OH, oxo, NH₂, NHC₁₋₆ alkyl, and N(C₁₋₆ alkyl)₂;
m is an integer selected from 0, 1, 2, and 3;
n is an integer selected from 0, 1, 2, 3, and 4; and
the membrane has a CO₂/CH₄ selectivity of from about 20 to about 40 at a temperature of 25 to 55 °C and a pressure of 200 to 1200 psi.

22. The membrane of claim 21, wherein the polyimide polymer of the membrane comprises a copolymer having a structure of Formula (II): 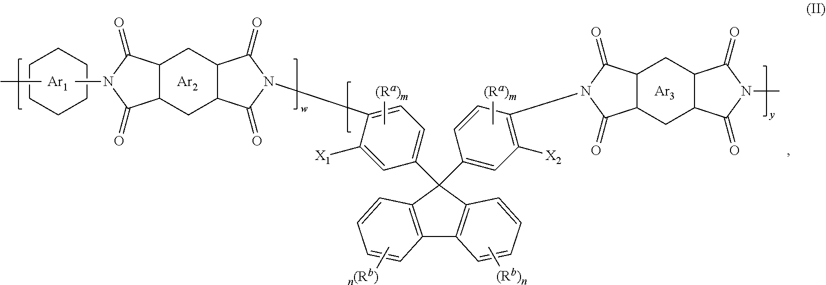
wherein Ar₁, Ar₂, and Ar₃ independently comprise aromatic moieties, bridging moieties, or both.

23. The method of claim 21, wherein the polyimide polymer of the membrane comprises a homopolymer having a structure of Formula (III): 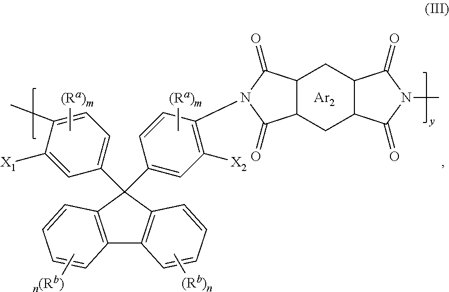
wherein Ar₁ comprises an aromatic moiety or a bridging moiety.

24. The method of claim 21, wherein the polyimide polymer of the membrane comprises a copolymer having a structure of Formula (III-A), (III-A), or (III-C):

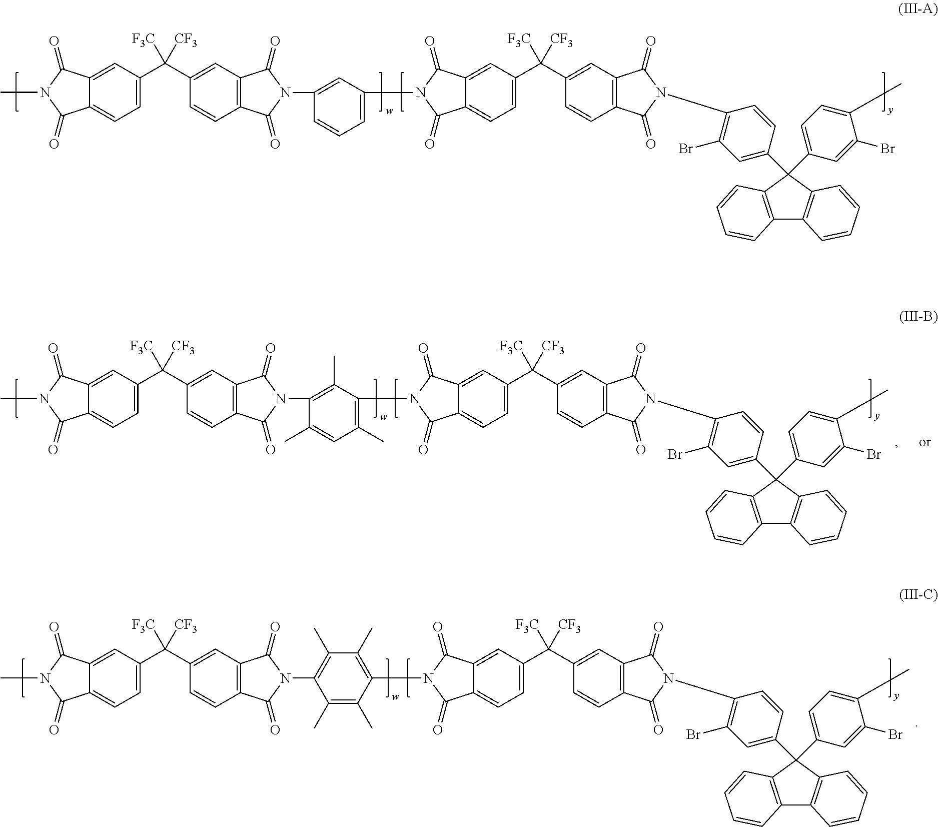

25. The method of claim 21, wherein the polyimide polymer of the membrane comprises a homopolymer having a structure of Formula (IV): [chemical structure]
